(19) United States
(12) Patent Application Publication
(10) Pub. No.: US 2026/0115471 A1
(43) Pub. Date: Apr. 30, 2026

(54) SYSTEMS AND METHODS FOR DELIVERING NEUROSTIMULATION TO INCREASE BLOOD GLUCOSE UPTAKE IN A PATIENT

(71) Applicant: Nevro Corp., Redwood City, CA (US)

(72) Inventor: Kerry Bradley, Glendale, CA (US)

(21) Appl. No.: 19/367,104

(22) Filed: Oct. 23, 2025

Related U.S. Application Data
(60) Provisional application No. 63/711,578, filed on Oct. 24, 2024.

(51) Int. Cl. A61N 1/36

(52) U.S. Cl. CPC A61N 1/36062; A61N 1/36003; A61N 1/36139; A61N 1/3615; A61N 1/36171; A61N 1/36178

(57) ABSTRACT

The present technology is directed to managing blood glucose levels in patients and associated systems and methods. For example, the present technology provides a method for decreasing blood glucose levels in patients by delivering electrical signals to a spinal column of the patient via an implanted signal delivery device. The stimulation induces motor contractions in the lower extremity or abdomen, thus causing glucose uptake from the bloodstream in an insulin-independent manner.

### CROSS-REFERENCE TO RELATED APPLICATIONS

The present application claims priority to U.S. Provisional Ser. No. 63/711,578, filed on Oct. 24, 2024, which is incorporated herein by reference in its entirety.

### TECHNICAL FIELD

The present technology is directed generally to methods and systems for treating non-insulin dependent diabetes mellitus (NIDDM) and/or decreasing blood glucose levels in a patient in need thereof by applying electrical stimulation to a target neural population located within the patient's spinal cord.

### BACKGROUND

Neurological stimulators have been developed to treat pain, movement disorders, functional disorders, spasticity, cancer, cardiac disorders, and various other medical conditions. Neurological stimulation systems generally have a signal generator and one or more implantable leads that deliver electrical pulses to neurological tissue or muscle tissue. For example, several neurological stimulation systems for spinal cord stimulation (SCS) have cylindrical leads that include a lead body with a circular cross-sectional shape and one or more conductive rings (e.g., contacts) spaced apart from each other at the distal end of the lead body. The conductive rings operate as individual electrodes and, in many cases, the SCS leads are implanted percutaneously through a needle inserted into the epidural space, with or without the assistance of a stylet. In other systems, the electrodes are carried by a paddle that is implanted via a laminotomy.

While the foregoing stimulators and treatments have proven beneficial in many instances, there remains a significant need in the medical community for improved therapies that can address metabolic diseases, such as diabetes generally, and more particularly, non-insulin dependent diabetes mellitus (NIDDM).

### BRIEF DESCRIPTION OF THE DRAWINGS

FIG. 1 is a partially schematic illustration of an implantable spinal cord modulation system positioned at a patient's spine to deliver electrical signals in accordance with some embodiments of the present technology.

FIG. 2A is a partially schematic, cross-sectional illustration of a patient's spine, illustrating representative locations for implanted lead bodies in accordance with some embodiments of the present technology.

FIG. 2B is a partially schematic, cross-sectional illustration of a patient's spine, illustrating further representative locations for implanted lead bodies in accordance with embodiments of the present technology.

FIG. 3 is a schematic illustration of a representative lead body suitable for providing modulation to a patient in accordance with some embodiments of the present technology.

FIG. 4 is a flowchart of a method of treating a patient in accordance with some embodiments of the present technology.

FIG. 5 is a flowchart of another method of treating a patient in accordance with some embodiments of the present technology.

FIG. 6 is a graph showing additional clinical data from Applicant's clinical study investigating the use of SCS for managing blood glucose levels, in accordance with embodiments of the present technology.

FIG. 7 is a graph showing certain clinical data from Applicant's clinical study investigating the use of SCS for managing blood glucose levels, in accordance with embodiments of the present technology.

FIG. 8 is a high-level block diagram illustrating an example AI system, in accordance with embodiments of the present technology.

FIG. 9 is a block diagram that illustrates an example of a computer system in which at least some operations described herein can be implemented.

### DETAILED DESCRIPTION

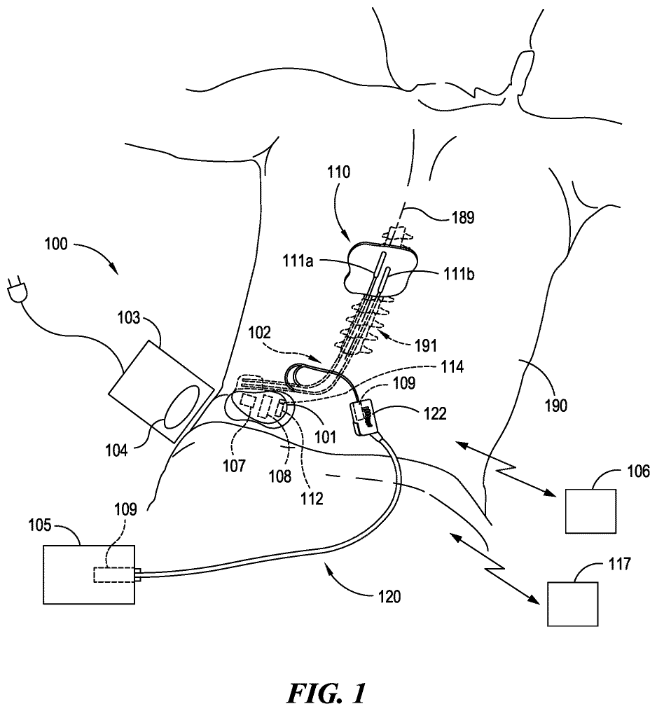

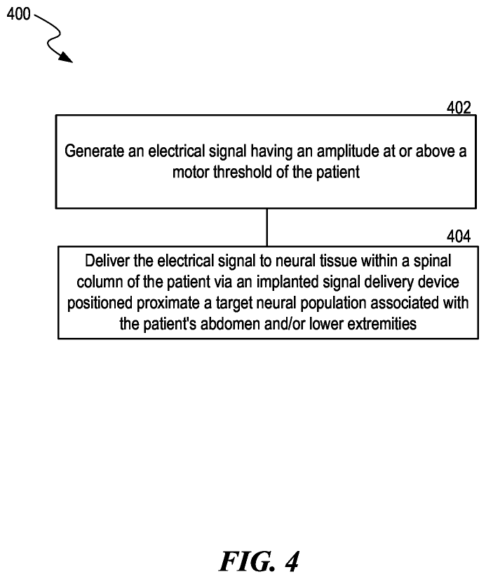

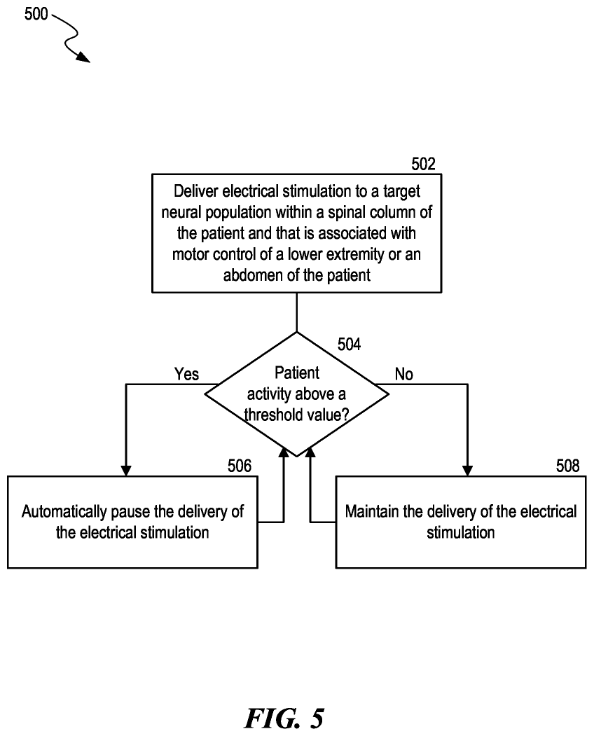

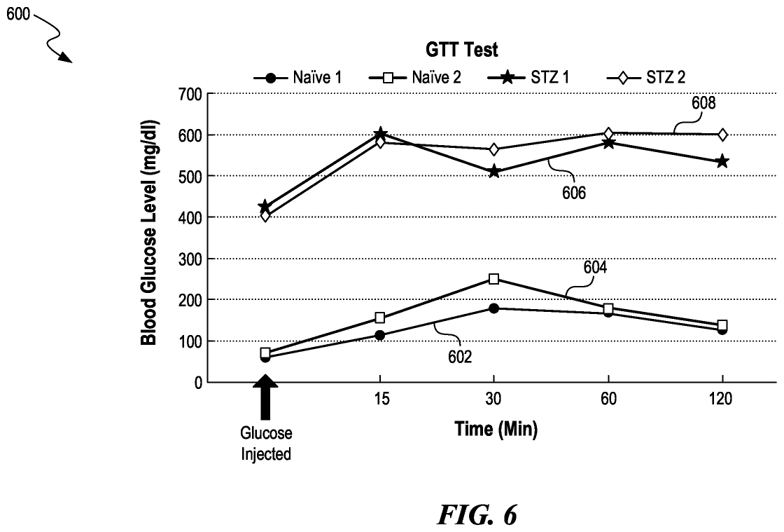

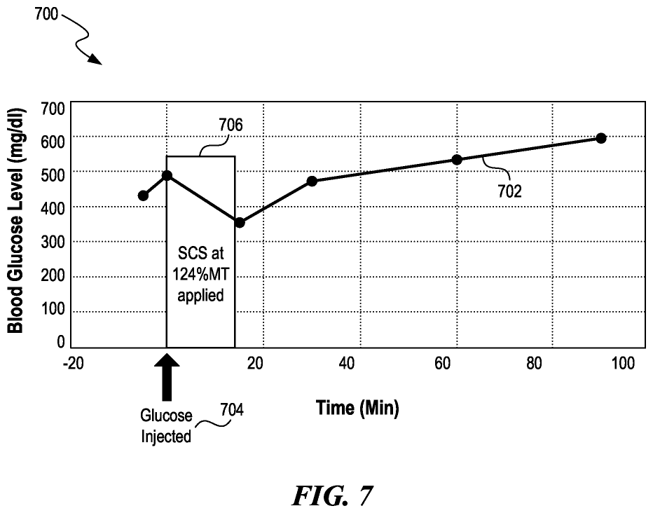

This Detailed Description includes the following headers and sections, which are provided for convenience only and do not affect the scope or meaning of the claimed present technology:
Definitions of selected terms are provided under Heading 1.0 (“Definitions”);
General aspects of the present technology are described below under Heading 2.0 (“Overview of Present Technology”);
Representative treatment systems and their characteristics are described under Heading 3.0 (“System Characteristics”) with reference to FIGS. 1-3;
Representative embodiments of the present technology are described under Heading 4.0 (“Representative Embodiments of the Present Technology”) with reference to FIGS. 4 and 5, including use of spinal cord stimulation (SCS) for managing blood glucose levels and associated methods described under subheading A, and maintenance stimulation schedules and associated method described under subheading B;
Representative stimulation parameters of the present technology are described under Heading 5.0 (“Representative Stimulation Parameters”);
Representative clinical applications for the present technology are described under Heading 6.0 (“Representative Clinical Applications”);
Clinical data obtained in accordance with the present technology is described under Heading 7.0 (“Clinical Data”) with reference to FIGS. 6 and 7;
Representative computing systems used to determine the stimulation parameters of the present technology are described under Heading 8.0 (“Representative Systems Used to Determine Stimulation Parameters”); and
Representative examples are described under Heading 9.0 (“Representative Examples”).

### 1.0 Definitions

Unless otherwise stated, the terms “generally,” “about,” and “approximately” refer to values within 10% of a stated value. For example, the use of the term “about 100” refers to a range of 90 to 110, inclusive. In instances in which relative terminology is used in reference to something that does not include a numerical value, the terms are given their ordinary meaning to one skilled in the art.

As used herein, and unless otherwise noted, the terms “modulate,” “modulation,” “stimulate,” and “stimulation” refer generally to electrical signals that have an inhibitory, excitatory, and/or other effect on a target neural population. Accordingly, a spinal cord “stimulator” can have an inhibitory effect and/or an excitatory effect on certain neural populations. Moreover, the use of the terms “suppress” and “inhibit” in relation to an electrical signal's effect on a neuron refers to a reduction in the neuron's firing rate relative to the neuron's baseline firing rate in the absence of the electrical signal, and does not necessarily refer to a complete elimination of action potentials in the neuron. The “baseline” firing rate can refer to the neuron's spontaneous firing rate and/or the firing rate of the neuron in response to an external stimulus other than the therapy signal.

As used herein, the terms “neuromodulation signal”, “electrical therapy signal,” “electrical signal,” “therapy signal,” “signal,” and other associated terms are used interchangeably and generally refer to an electrical signal that can be characterized by one more parameters, such as frequency, pulse width, and/or amplitude.

As used herein, the term “paresthesia-free” when used to describe an electrical signal refers to an electrical signal that does not produce paresthesia when delivered to a patient. Paresthesia-free signals may have combinations of frequency, pulse widths, amplitudes, and/or pulse dosing rates that cause the signal to be below a patient's perception threshold. For example, paresthesia-free electrical signals may have a frequency of between about 0.01 Hz and about 1 MHz, or between about 1.2 kHz and 500 kHz, or between about 1.2 kHz and about 100 kHz. As another example, paresthesia-free electrical signals may be delivered in discrete bursts, separated by quiescent periods in which the electrical signal is not delivered. As yet another example, paresthesia-free electrical signals may have a “long” pulse width of between about 5 milliseconds and about 25 seconds with an amplitude below an activation threshold of a target neural population. Additional examples of paresthesia-free electrical signals, including those mentioned above, are described in U.S. Patent Application Publication Nos. US2010/0274314, US2021/0228881, and US2022/0401730, and International Patent Application No. PCT/US2024/028654, each of the which is incorporated by reference herein.

As used herein, the term “pulse width” refers to the width of any phase of a repeating pulse, such as the portion of a pulse at a given polarity, unless explicitly described otherwise. For example, the use of the term pulse width with respect to a signal having bi-phasic pulses can refer to the duration of an anodic pulse phase or a cathodic pulse phase. The use of the term pulse width with respect to a signal having monophasic pulses can refer to the duration of the monophasic pulse phase.

As used herein, the term “pulse dosing” refers to repeatedly alternating between a first period in which pulses of an electrical signal are actively delivered and a second period in which the pulses are not delivered (e.g., a quiescent period). The pattern of alternating between the first (active) period and the second (quiescent) period is repeated for the “duration” the therapy is being applied for. The term “pulse dosing rate” or equivalents therefore refer to the percentage of the first period compared to the sum of the first period and the second period. For example, an electrical signal may be delivered continuously for 30 seconds (i.e., the first period equals 30 seconds), followed by a 30 second quiescent period (i.e., the second period equals 30 seconds). Such signal has a pulse dosing rate of 50%. In general, for purposes of pulse dosing, the second (quiescent) period is typically less than about 60 minutes, and more typically less than about 30 minutes, less than about 15 minutes, less than about 5 minutes, less than about 1 minute, or less than about 30 seconds. As described below, pulse dosing rates in accordance with the present technology can range from about 1% to about 100%.

In contrast, the terms “bolus” and “stimulation session” refer to a discrete period during which neurostimulation therapy is being applied to a patient, followed by a relatively prolonged off-period during which no neurostimulation therapy is applied to a patient. Of note, the neurostimulation therapy may be “pulsed dosed” during the discrete period during which neurostimulation therapy is being applied. For example, a “bolus” or “stimulation session” of one hour may include delivering an electrical stimulation at a pulse dose rate of 50%, such as alternating between 30 second active periods and 30 second quiescent periods for the one hour bolus period. In general, a bolus or stimulation session is typically followed by an off-period of greater duration than any quiescent period associated with pulse dosing. For example, a bolus or stimulation session is typically followed by an off-period of at least 1 hour, and more typically by an off-period of at least 2 hours, at least 3 hours, or at least 4 hours.

As used herein, “type II diabetes (T2D)” or “non-insulin dependent diabetes mellitus (NIDDM)” refers to a disease of impaired glucose metabolism and/or impaired insulin-dependent regulation of glucose levels. While NIDDM is systemic, it affects the liver, pancreas, kidneys, stomach, adrenal glands, heart, blood vessels, nerves, eyes, feet, hands, skin, and brain more so than other organs. The systems and methods of the present technology are configured to treat NIDDM.

“Treating” or “treatment” as used herein with regard to NIDDM refers to preventing progression and/or onset of NIDDM, ameliorating, reducing, eliminating, suppressing, and/or alleviating NIDDM, and/or one or more of the symptoms associated with NIDDM, generating a complete or partial regression of NIDDM, or any suitable combination thereof. “Treatment” also refers to reducing a patient's HbA1c levels and/or increasing blood glucose uptake from a bloodstream of a patient.

As used herein, the term “HbA1c” refers to hemoglobin A1c, a glycated form of hemoglobin.

As used herein, the term “blood glucose uptake” or “glucose uptake” refers to the physiological process by which glucose is transported from the bloodstream into cells of a patient via glucose transporters (GLUTs) located on the cell membrane and/or other mechanisms.

As used herein, the term “blood glucose abnormality” refers to NIDDM and/or higher than normal HbA1c levels, higher than normal fasting blood glucose levels, higher than normal oral glucose tolerance tests, and/or a state of persistent hyperglycemia.

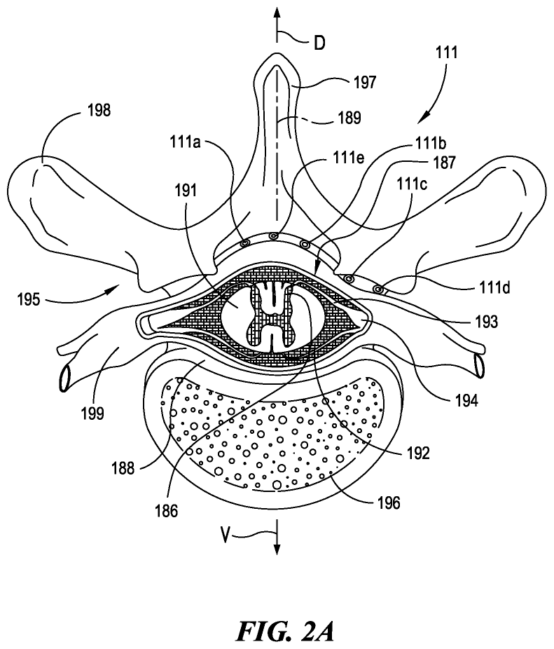

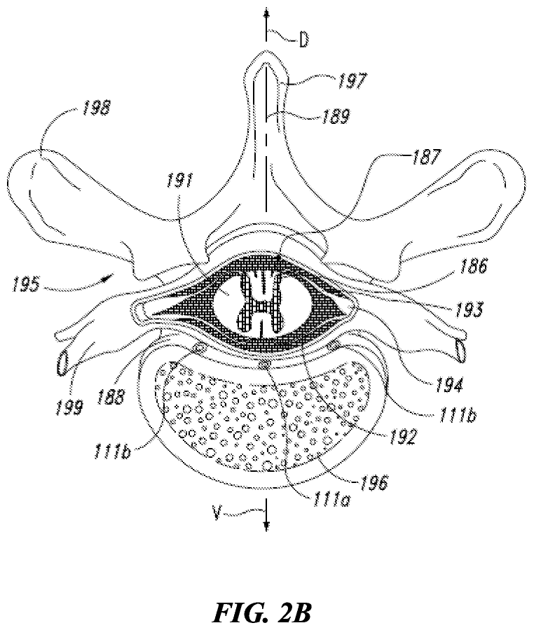

As used herein, “proximate a spinal cord region” refers to the placement of a signal delivery element or device such that it can deliver electrical stimulation to a neural population located in the spinal cord and/or within the spinal canal. For example, “proximate a spinal cord region” includes, but is not limited to, the relative lead positions described and shown in FIGS. 2A and 2B, as well as other positions not expressly described herein.

As used herein, “proximate a target neural population” refers to the placement of a signal delivery element such that it can deliver electrical stimulation to the target neural population. For example, if the target population includes neurons in the spinal cord at a given vertebral level, “proximate the target neural population” includes, but is not limited to, the relative lead positions described and shown in FIG. 2A and FIG. 2B at the given vertebral level, as well as other positions not expressly described herein. As another example, if the target population includes neurons in the patient's cortex (e.g., motor cortex), “proximate the target neural population” includes, but is not limited to, leads positioned in or on the patient's cortex or near deep brain structures.

### 2.0 Overview of the Present Technology

Non-insulin dependent diabetes mellitus (NIDDM), also known as type II diabetes (T2D), is a chronic condition characterized by insulin resistance and relative insulin deficiency, leading to elevated blood glucose levels. Many patients with NIDDM have limited options to treat the patient's elevated blood glucose levels. Conventional approaches such as diet, exercise, and pharmacologic interventions are challenged by drug side effects, behavioral limitations, and/or cardiovascular risks. For example, dietary modifications (e.g., low-carbohydrate diets, calorie restriction) may be difficult for patients to maintain consistently. Further, exercise (e.g., aerobic activities, resistance training) may be limited by physical or behavioral constraints (e.g., mobility issues, lack of motivation), and thus pose as an unreliable option for some individuals. Similarly, pharmacologic treatments (e.g., metformin, sulfonylureas) may be accompanied by undesirable side effects (e.g., gastrointestinal issues, hypoglycemia) and may pose cardiovascular risks, further complicating the management of blood glucose in a patient with NIDDM.

The present technology is directed generally to spinal cord stimulation and associated systems and methods for treating NIDDM, including by reducing the patient's elevated blood glucose levels associated with the patient's NIDDM. In particular, the spinal cord stimulation therapies described herein may induce motor contractions in specific regions of the body, such as the lower extremities and abdomen, to facilitate blood glucose uptake from the bloodstream in an insulin-independent manner. As described below, in some embodiments this is accomplished by programming a signal generator to generate and deliver electrical signals to a patient's spinal cord. The electrical signals may be administered near and/or above the motor threshold to effectively induce the desired motor contractions.

Without intending to be bound by theory, the present technology is expected to improve glucose management in an insulin independent manner (e.g., without the need for insulin), produce reduced side effects, and/or enable tailoring of the treatment to individual patient needs through programmable and adaptive stimulation features. The spinal cord stimulation therapies described herein can be used to address a variety of conditions by reducing glucose levels in addition to addressing NIDDM, such as insulin-dependent diabetes mellitus (IDDM) (e.g., by increasing glucose uptake in an insulin-independent manner to complement insulin therapy), gestational diabetes mellitus (GDM) (e.g., by maintaining stable glucose levels during pregnancy), or other blood glucose-related conditions.

Specific details of certain embodiments of the disclosure are described below with reference to methods for modulating one or more target neural populations (e.g., nerves) or sites of a patient, and associated implantable structures for providing the modulation. Although selected embodiments are described below with reference to modulating the dorsal column, dorsal horn, dorsal root, dorsal root entry zone, ventral column, ventral horn, and/or other particular regions of the spinal column, the modulation may in some instances be directed to other neurological structures and/or target neural populations of the spinal cord and/or other neurological tissues throughout the body. For example, some embodiments may include modulating brain tissue, including the cortex (e.g., motor cortex) and/or deep brain structures. As another example, some embodiments may include modulating peripheral nervous tissue, such as dorsal root ganglion and/or or peripheral nerves that directly innervate a target muscle. Some embodiments can have configurations, components, or procedures different than those described in this section, and other embodiments may eliminate particular components or procedures. A person of ordinary skill in the relevant art, therefore, will understand that the present disclosure may include other embodiments with additional elements, and/or may include other embodiments without several of the features shown and described below with reference to FIGS. 1-9.

### 3.0 System Characteristics

FIG. 1 schematically illustrates a representative patient therapy system 100 for treating a patient's motor, sensory, and/or other functioning, arranged relative to the general anatomy of the spinal column 191 of a patient 190. The system 100 can include a signal generator 101 (e.g., an implanted or implantable pulse generator or IPG), which can be implanted subcutaneously within a patient 190 and coupled to one or more signal delivery elements or devices 110. The signal delivery elements or devices 110 can be implanted within the patient 190, at or off the patient's spinal cord midline 189. The signal delivery elements 110 carry features for delivering therapy to the patient 190 after implantation. The signal generator 101 can be connected directly to the signal delivery devices 110, or it can be coupled to the signal delivery devices 110 via a signal link, e.g., a lead extension 102. In some embodiments, the signal delivery devices 110 can include one or more elongated lead(s) or lead body or bodies 111 (identified individually as a first lead 111a and a second lead 111b). As used herein, the terms signal delivery device, signal delivery element, lead, and/or lead body include any of a number of suitable substrates and/or supporting members that carry electrodes/devices for providing therapy signals to the patient 190. For example, the lead or leads 111 can include one or more electrodes or electrical contacts that deliver electrical signals into the patient's tissue, e.g., to provide for therapeutic relief. In some embodiments, the signal delivery elements 110 can include structures other than a lead body (e.g., a paddle) that also deliver electrical signals and/or other types of signals to the patient 190, e.g., as disclosed in U.S. Patent Application Publication No. 2018/0256892, incorporated herein by reference in its entirety. In some embodiments, paddles can be more suitable for patients with stenosis or other indications that compromise the epidural space and preclude the percutaneous delivery of cylindrical leads.

In some embodiments, one signal delivery device can be implanted on one side of the spinal cord midline 189, and a second signal delivery device can be implanted on the other side of the spinal cord midline 189. For example, the first and second leads 111a, 111b shown in FIG. 1 can be positioned just off the spinal cord midline 189 (e.g., about 1 mm offset) in opposing lateral directions so that the two leads 111a, 111b are spaced apart from each other by about 2 mm. In some embodiments, the leads 111 can be implanted at a vertebral level ranging from, for example, about T1 to about T12, or from about T4 to about T12. In some embodiments, one or more signal delivery devices can be implanted at other vertebral levels, e.g., as disclosed in U.S. Pat. No. 9,327,121, incorporated herein by reference in its entirety. In other embodiments, one or more leads 111 can be implanted at or proximate other target neural structures, including brain tissue, peripheral nerves, etc.

The signal generator 101 can transmit signals (e.g., electrical signals) to the signal delivery elements 110 that excite, inhibit, downregulate and/or suppress target nerves. The signal generator 101 can include a machine-readable (e.g., computer-readable or controller-readable) medium containing instructions for generating and transmitting suitable therapy signals, such as to perform the methods described below with respect to FIGS. 4 and 5. The signal generator 101 and/or other elements of the system 100 can include one or more processor(s) 107, memory unit(s) 108, and/or input/output device(s) 112. Accordingly, the process of providing modulation signals, providing guidance information for positioning the signal delivery devices 110, establishing battery charging and/or discharging parameters, and/or executing other associated functions can be performed by computer-executable instructions contained by, on, or in computer-readable media located at the pulse generator 101 and/or other system components. Further, the pulse generator 101 and/or other system components can include dedicated hardware, firmware, and/or software for executing computer-executable instructions that, when executed, perform any one or more methods, processes, and/or sub-processes described herein and/or in the materials incorporated herein by reference. The dedicated hardware, firmware, and/or software also serve as “means for” performing the methods, processes, and/or sub-processes described herein. The signal generator 101 can also include multiple portions, elements, and/or subsystems (e.g., for directing signals in accordance with multiple signal delivery parameters), carried in a single housing, as shown in FIG. 1, or in multiple housings. For example, the signal generator can include some components that are implanted (e.g., a circuit that directs signals to the signal delivery device 110), and some that are not (e.g., a power source). The computer-executable instructions can be contained on one or more media that are implanted within the patient and/or positioned external to the patient, depending on the embodiment.

The signal generator 101 can also receive and respond to an input signal received from one or more sources. The input signals can direct or influence the manner in which the therapy, charging, and/or process instructions are selected, executed, updated, and/or otherwise performed. The input signals can be received from one or more sensors (e.g., an input device 112 shown schematically in FIG. 1 for purposes of illustration) that are carried by the signal generator 101 and/or distributed outside the signal generator 101 (e.g., at other patient locations) while still communicating with the signal generator 101. The sensors and/or other input devices 112 can provide inputs that depend on or reflect patient state (e.g., patient position, patient posture, and/or patient activity level), and/or inputs that are patient-independent (e.g., time). Still further details are included in U.S. Pat. No. 8,355,797, incorporated herein by reference in its entirety.

In some embodiments, the signal generator 101 and/or signal delivery devices 110 can obtain power to generate the therapy signals from an external power source 103. In some embodiments, the external power source 103 can bypass an implanted signal generator and generate a therapy signal directly at the signal delivery devices 110 (or via signal relay components). The external power source 103 can transmit power to the implanted signal generator 101 and/or directly to the signal delivery devices 110 using electromagnetic induction (e.g., RF signals). For example, the external power source 103 can include an external coil 104 that communicates with a corresponding internal coil (not shown) within the implantable signal generator 101, signal delivery devices 110, and/or a power relay component (not shown). In some embodiments, the external power source 103 can transmit power to the implanted signal generator 101 and/or directly to the signal delivery devices 110 in a generally continuous manner such that the system 100 can operate without an internal power source. The external power source 103 can be portable for ease of use.

In some embodiments, the implanted signal generator 101 can be omitted and the external power source 130 can be configured as an external signal generator that transmits power and/or electrical signals to the signal delivery devices 110 (e.g., via an implanted relay device; not shown). For example, the external power source 103 can either transmit the electrical signal itself to the signal delivery device or cause an electrical signal to be generated directly at the signal delivery devices 110 or at an implanted relay device (not shown). In such embodiments, the external power source 103 can be a wearable device that the patient wears while receiving therapy. In such embodiments, the patient only receives stimulation therapy while the wearable device is placed in an active state and is being worn by the patient. This is generally less invasive because such embodiments generally do not require an implanted signal generator 101, nor an implanted power storage device. In such embodiments, the external power source 103 may be wireless to enable patient mobility and/or accurate positioning of the external power source 103 during treatment. For example, the external power source 103 can include a rechargeable battery (not shown) that can be used to power the external power source 103 while in the active state, and recharged while in an inactive state.

In some embodiments, the signal generator 101 can obtain the power to generate therapy signals from an internal power source, in addition to or in lieu of the external power source 103. For example, the implanted signal generator 101 can include a non-rechargeable battery (e.g., a primary cell) or a rechargeable battery (e.g., a secondary cell) to provide such power. When the internal power source includes a rechargeable battery, the external power source 103 can be used to recharge the battery. The external power source 103 can in turn be recharged from a suitable power source (e.g., conventional wall power).

During at least some procedures, an external stimulator or trial modulator 105 can be coupled to the signal delivery elements 110, e.g., during an initial procedure, prior to implanting the signal generator 101. For example, a practitioner (e.g., a physician and/or a company representative) can use the trial modulator 105 to vary the modulation parameters provided to the signal delivery elements 110 in real time, and select optimal or particularly effective parameters. These parameters can include the location from which the electrical signals are emitted, as well as the characteristics of the electrical signals provided to the signal delivery devices 110. In some embodiments, input is collected via the external stimulator or trial modulator 105 and can be used by the clinician to help determine what parameters to vary. In a typical process, the practitioner uses a wireless connection or cable assembly 120 to temporarily connect the trial modulator 105 to the signal delivery device 110. The practitioner can test the effectiveness of the signal delivery devices 110 in an initial position. The practitioner can then disconnect the cable assembly 120 if needed (e.g., at a connector 122), reposition the signal delivery devices 110, and reapply the electrical signals. This process can be performed iteratively until the practitioner obtains the desired position for the signal delivery devices 110. Optionally, the practitioner can move the partially implanted signal delivery devices 110 without disconnecting the cable assembly 120. Furthermore, in some embodiments, the iterative process of repositioning the signal delivery devices 110 and/or varying the therapy parameters may not be performed.

The signal generator 101, the lead extension 102, the trial modulator 105 and/or the connector 122 can each include a receiving element 109. Accordingly, the receiving elements 109 can be patient implantable elements, or the receiving elements 109 can be integral with an external patient treatment element, device or component (e.g., the trial modulator 105 and/or the connector 122). The receiving elements 109 can be configured to facilitate a simple coupling and decoupling procedure between the signal delivery devices 110, the lead extension 102, the pulse generator 101, the trial modulator 105 and/or the connector 122. The receiving elements 109 can be at least generally similar in structure and function to those described in U.S. Patent Application Publication No. 2011/0071593, incorporated by reference herein in its entirety.

After the signal delivery elements 110 are implanted, the patient 190 can receive therapy via signals generated by the trial modulator 105 or via another external signal generator (e.g., the power source 103), generally for a limited period of time. During this time, the patient wears the trial modulator 105 outside the body. Assuming the trial therapy is effective or shows the promise of being effective, the practitioner then replaces the trial modulator 105 with the implanted signal generator 101, and programs the signal generator 101 with therapy programs selected based on the experience gained during the trial period. Optionally, the practitioner can also replace the signal delivery elements 110. In still further embodiments, the signal generator 101 can be implanted without first undergoing a trial period. Once the implantable signal generator 101 has been positioned within the patient 190, the therapy programs provided by the signal generator 101 can still be updated remotely via a wireless physician's programmer 117 (e.g., a physician's laptop, a physician's remote or remote device, etc.) and/or a wireless patient programmer 106 (e.g., a patient's laptop, patient's remote or remote device, etc.). Generally, the patient 190 has control over fewer parameters than does the practitioner. For example, the capability of the patient programmer 106 can be limited to starting and/or stopping the signal generator 101, selecting a pre-programmed therapy option, and/or adjusting the signal amplitude within a present amplitude range. The patient programmer 106 can be configured to accept inputs corresponding to pain relief, motor functioning and/or other variables, such as medication use. Accordingly, more generally, embodiments of the present technology include receiving patient feedback, via a sensor, that is indicative of, or otherwise corresponds to, the patient's response to the signal. Feedback includes, but is not limited to, motor, sensory, and verbal feedback. In response to the patient feedback, one or more signal parameters can be adjusted, such as frequency, pulse width, amplitude, or delivery location. In some embodiments, the patient programmer can be a network connected handheld computing device such as a smartphone, which can include a patient app that provides various functions such as remote programming, therapy selection, therapy tracking information such as current blood glucose levels, presence or absence of stimulation-induced muscle contractions, perceived intensity of stimulation-induced muscle contractions, etc., for use by the clinician and/or software in the app to optimize therapy.

FIGS. 2A and 2B are cross-sectional illustrations of the spinal cord 191 and an adjacent vertebra 195 (based generally on information from Crossman and Neary, “Neuroanatomy,” 1995 (published by Churchill Livingstone)), along with multiple leads 111 (shown as leads 111a-111e) implanted at representative locations. For purposes of illustration, multiple leads 111 are shown in FIGS. 2A and 2B implanted in a single patient. In addition, for purposes of illustration, the leads 111 are shown as elongated leads however, leads 111 can be paddle leads. In actual use, any given patient will likely receive fewer than all the leads 111 shown in FIG. 2A and FIG. 2B.

As shown in FIGS. 2A and 2B, the spinal cord 191 is situated within a vertebral foramen 188, between a ventrally located ventral body 196 and a dorsally located transverse process 198 and spinous process 197. Arrows V and D identify the ventral and dorsal directions, respectively. The spinal cord 191 itself is located within the dura mater 199, which also surrounds portions of the nerves exiting the spinal cord 191, including the ventral roots 192, dorsal roots 193, and dorsal root ganglia 194. The dorsal roots 193 enter the spinal cord 191 at the dorsal root entry region 187, and communicate with dorsal horn neurons located at the dorsal horn 186.

In some embodiments, as shown in FIG. 2A, one or more leads 111 can be positioned on a dorsal side of the patient's spinal cord 191. In particular, in FIG. 2A the first and second leads 111a, 111b are positioned just off the spinal cord midline 189 (e.g., about 1 mm offset) in opposing lateral directions so that the two leads 111a, 111b are spaced apart from each other by about 2 mm, as discussed above. In some embodiments, a lead or pairs of leads can be positioned at other locations, e.g., toward the outer edge of the dorsal root entry region 187 as shown by a third lead 111c in FIG. 2A, or at the dorsal root ganglia 194, as shown by a fourth lead 111d in FIG. 2A, or approximately at the spinal cord midline 189, as shown by a fifth lead 111e in FIG. 2A. One or more of the leads 111a-111e can deliver neurostimulation to various targets within the spinal cord spinal cord, including, but not limited to, the dorsal column, the dorsal horn, the intermediolateral nucleus, and/or other neurons within any of laminae I-X of the spinal cord.

In some embodiments, as shown in FIG. 2B, one or more leads 111 can be positioned on a ventral side of the patient's spinal cord 191. In particular, in FIG. 2B, a single first signal delivery device 111a is positioned at a ventral location within the vertebral foramen 188 (e.g., within the patient's spinal canal), at or approximately at the spinal cord midline 189. Similarly, in another embodiment shown in FIG. 2B, one or more signal delivery devices 111b are positioned off the spinal cord midline 189, laterally or bilaterally. From these locations, the signal delivery device(s) 111 can direct therapeutic signals to ventral neural populations of the spinal cord 191 itself, or to neural populations in the region of the spinal cord, but off the spinal cord itself, e.g., the laterally-positioned ventral roots 192.

In general, the signal delivery device(s) 111 (and more particularly, the electrodes carried by the signal delivery device(s) 111) are positioned proximate to (e.g., within 0.5 mm-10 mm of) the target neural population. The specific location within the foregoing range can be selected by the practitioner to produce the desired therapeutic outcome and/or to avoid unwanted side effects. In still further embodiments, one or more signal delivery devices can be positioned laterally or bilaterally at other locations.

In some embodiments, the devices and systems of the present technology include features other than those shown herein. For example, regardless of whether the leads are positioned dorsally or ventrally, one lead 111 to six leads 111 can be positioned generally end-to-end at or near the patient's midline M and span vertebral levels from about C2 to about T12, or from about T4 to about T12, or from about T8 to about T12. In some embodiments, two, three, or four leads 111 are positioned end-to-end at or near the patient's midline from T4 to T12. In some embodiments, the leads 111 and/or other signal delivery devices can have locations other than those expressly shown herein. For example, one or more signal delivery devices can be positioned at the dorsal side of the spinal cord 191. In addition, the devices and systems of the present technology can include more than one internal stimulator and/or more than one external stimulator that can be configured for wireless stimulation, such as by using electromagnetic waves.

Several aspects of the technology are embodied in computing devices, e.g., programmed/programmable pulse generators, controllers and/or other devices. The computing devices on/in which the described technology can be implemented can include one or more central processing units, memory, input devices (e.g., input ports), output devices (e.g., display devices), storage devices, and network devices (e.g., network interfaces). The memory and storage devices are computer-readable media that can store instructions that implement the technology. In some embodiments, the computer readable media are tangible media. In some embodiments, the data structures and message structures can be stored or transmitted via an intangible data transmission medium, such as a signal on a communications link. Various suitable communications links can be used, including but not limited to a local area network and/or a wide-area network.

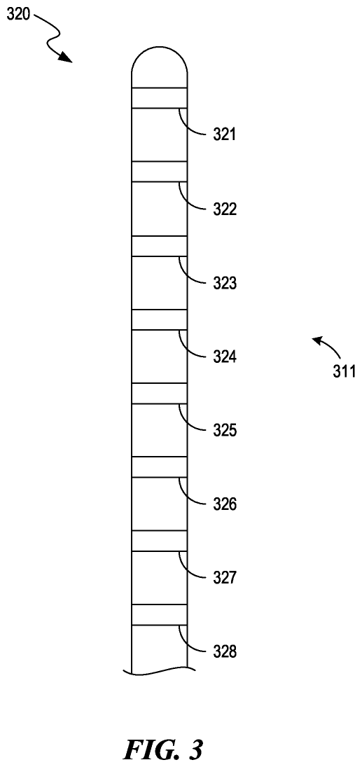

FIG. 3 is a partially schematic illustration of a representative lead body 311 that can be used to apply modulation to a patient in accordance with any of the foregoing embodiments. In general, the lead body 311 includes a multitude of electrodes or contacts 320. When the lead body 311 has a circular cross-sectional shape, as shown in FIG. 3, the contacts 320 can have a generally ring-type or segmented shape and can be spaced apart axially along the length of the lead body 311. In a particular embodiment, the lead body 311 can include eight contacts 320, identified individually as first, second, third. eighth contacts 321, 322, 323. 328, although in other embodiments the lead body 311 can include fewer or more electrodes, such as between 1 electrode and 64 electrodes. In general, one or more of the contacts 320 are used to provide signals, and another one or more of the contacts 320 provide a signal return path. Accordingly, the lead body 311 can be used to deliver monopolar modulation (e.g., if the return contact is spaced apart significantly from the delivery contact), or bipolar modulation (e.g., if the return contact is positioned close to the delivery contact and in particular, at the same target neural population as the delivery contact). In still further embodiments, the pulse generator 101 (FIG. 1) can operate as a return contact for monopolar modulation.

### 4.0 Representative Embodiments of the Present Technology

The representative systems described above under Heading 3.0 and elsewhere herein can be used to carry out the methods described under Heading 4.0 and elsewhere herein.

Glucose is transported from a subject's bloodstream to a subject's cells via a variety of mechanisms to provide energy to the cells. For example, insulin is a hormone that binds to receptors on the cell surface, which triggers the translocation of glucose transporter proteins (GLUT4) to the cell membrane, which in turn allows glucose to passively diffuse into the cell through the GLUT4 transporters. This mechanism can be referred to as insulin-dependent glucose uptake. However, these GLUT4 transporters can be translocated to the cell membrane via other mechanisms as well that do not require insulin. For example, when muscles contract, the muscle cells increase their demand for energy, which is supplied by glucose in the bloodstream. Thus, muscle contraction can also induce the translocation of the GLUT4 transporters to the cell membrane to promote the uptake of glucose into muscle cells to provide the necessary energy for contraction. Of note, this translocation and subsequent glucose uptake can occur independent of insulin, and can therefore be referred to as insulin-independent glucose uptake. By increasing glucose uptake, muscle contractions thus lower blood glucose levels in the patient and improve overall glucose homeostasis. The present technology utilizes the effects of motor contractions on glucose uptake to manage conditions such as NIDDM or other conditions with impaired insulin signaling. For example, many embodiments of the present technology include treating NIDDM and/or reducing HbA1c/blood glucose levels by applying electrical stimulation to induce motor contractions in the patient, thereby stimulating the uptake of glucose in an insulin-independent manner.

For example, electrical stimulation can be delivered to the patient's spinal cord region at or near one or more of the patient's thoracic vertebrae T1-T12 and/or lumbar vertebrae L1-L5 to induce motor contractions in the patient. The vertebral level to be stimulated can be selected based on which muscles are being targeted for contraction. For example, stimulating at spinal segments T9-T12 generally corresponds to muscle contractions in the abdomen and lower back, whereas stimulating at spinal segments L1-L5 generally corresponds to muscle contractions in the lower extremities. In other embodiments, electrical stimulation can be delivered to other locations within the patient to induce muscle contractions. For example, stimulation can be delivered to the motor cortex of the patient's brain and/or peripheral nerves.

Without intending to be bound by any particular theory, delivering electrical signals to induce muscle contractions may improve glucose tolerance in patients having elevated blood glucose levels (e.g., NIDDM) by increasing the uptake of glucose from the bloodstream of the patient. The present technology provides methods and devices for treating the patient's blood glucose abnormalities (e.g., NIDDM), and/or reducing the patient's blood glucose/HbA1c levels. Methods and systems for treating the patient's blood glucose abnormality by applying electrical signals to thoracic and/or lumbar neural populations, are discussed immediately below.

### A. Management of Blood Glucose Levels and Associated Methods

As set forth above, the present technology includes stimulation schedules/regimens that are expected to reduce blood glucose and/or HbA1c levels in a patient. This is accomplished by applying electrical stimulation/signals at or above the motor threshold of the patient to the spinal cord or another suitable target nerve of a patient. Additionally, the present technology includes stimulation schedules/regimens that target specific periods when the patient is most likely to benefit from the therapy (e.g., when the patient is in a sedentary state, when the patient is prandial or post-prandial, etc.).

FIG. 4 is a block diagram illustrating a method 400 of treating a patient in accordance with some embodiments of the present technology. Some or all of the operations in the method 400 can be performed by a processor executing instructions stored on one or more elements of a patient treatment system, including the patient treatment system 100 described with reference to FIG. 1. This approach can be used with any of the signal delivery modalities described herein (e.g., signal delivered via one signal device or more than one, signals delivered to one target neural population or more than one, etc.).

The method 400 can begin at block 402 by generating an electrical signal having an amplitude at or above a motor threshold of the patient. The electrical signal can be generated by a signal generator (e.g., the implantable signal generator 102 of FIG. 1). For example, the signal generator can be programmed with specific instructions to produce the desired electrical signal, and subsequently stored in a memory element of the implanted signal delivery device 110. The motor threshold can be the minimum amplitude of electrical stimulation required to elicit a motor response, such as a muscle contraction, in the patient. To determine the motor threshold, the patient can receive electrical signals with gradually increasing amplitudes. For example, the patient can receive a series of test pulses delivered through the implanted signal delivery device 110. The amplitude can be incrementally increased until a visible or measurable motor response, such as a muscle twitch or contraction, is observed. The amplitude can be determined as the motor threshold for that specific patient. Motor contractions induced by the electrical stimulation can be detected using electromyography (EMG) sensors placed on the lower extremity or the abdomen of the patient. EMG sensors can measure the electrical activity produced by skeletal muscles and provide a quantitative assessment of muscle response to stimulation. When the electrical signal reaches the motor threshold, the EMG sensors can detect an increase in electrical activity. Additionally or alternatively, motor contractions can be detected via patient feedback and/or through visually observing twitching or contraction of the muscle.

The electrical signal generated at block 402 can have various signal parameters, including frequency, pulse width, and amplitude, each of which can be adjusted depending on the therapeutic effect. For example, the signal can have a frequency within a frequency range of from about 0.1 Hz to about 100 kHz. In some embodiments, the frequency can be within a first, e.g., low, frequency band (e.g., 0.1-2 Hz), a second, e.g., middle, frequency band (e.g., 10-50 Hz), and/or a third, e.g., high, frequency band (e.g., 500 Hz-10 kHz). In some embodiments, the signal can have a pulse width within a pulse width range of from about 20 microseconds to about 1 second, such as from about 20 microseconds to about 200 microseconds or from about 200 microseconds to about 1 millisecond. In some embodiments, the signal can have an amplitude from about 0.1 mA to about 20 mA, such as from about 1 mA to about 5 mA, about 5 mA to about 10 mA, or from about 10 mA to about 15 mA. The electrical signal can also have any of the signal parameters discussed below under Heading 5.0 and elsewhere herein. The signal parameters can be selected based on the specific therapeutic goals, the patient's physiological responses, and/or other factors (e.g., available power levels).

In some embodiments, at least a portion of the electrical signals are paresthesia-free electrical signals that do not induce a sensation of paresthesia when administered to the patient. That is, the patient generally cannot sense whether the signal is being actively applied other than via the induced muscle contractions. The paresthesia-free signals may include different types of paresthesia-free signals, including, but not limited to, high frequency, paresthesia-free signals and/or low frequency, paresthesia-free signals. In other embodiments, the one or more of the electrical signals delivered during operation of the method 400 can be paresthesia-producing signals.

The method 400 can continue at block 404 by delivering the electrical signal to neural tissue within the spinal column of the patient via an implanted signal delivery device positioned proximate a target neural population associated with the patient's abdomen and/or lower extremities. The target neural population can include neural tissue within the spinal column located at and/or extending through any target vertebral level, such as T8, T9, T10, T11, T12, L1, L2, L3, L4, and/or L5 vertebral levels. The electrical stimulation can be delivered through electrodes implanted in the epidural space of the spinal column. The delivery of the electrical signal to the neural tissue within the spinal column can excite/activate some of the neurons within the target neural population to induce motor contractions in the patient's abdomen and/or lower extremities. The motor contractions can facilitate glucose uptake by the muscles, thereby helping to lower blood glucose levels in patients with conditions such as NIDDM in an insulin-independent manner. In some embodiments, the effectiveness of the stimulation can be monitored using various sensors that measure physiological parameters of the patient such as muscle activity, blood glucose levels, and patient-reported outcomes. In some embodiments, the implanted signal delivery device can be remotely monitored and controlled.

The electrical signal can be delivered in a continuous or non-continuous manner. For example, in some embodiments the electrical signal can be delivered according to a pulse-dosing regimen in which the signal is delivered for about 1 second to about 10 seconds, followed by about 1 second to about 10 seconds of no stimulation. Other representative pulse-dosing patterns include 0.5 seconds to 90 seconds of stimulation followed by 0.5 seconds to 90 seconds of no stimulation. Without intending to be bound by theory, providing stimulation in bursts may reduce the likelihood the patient habituates to the electrical signal. In other embodiments, the electrical signal can be delivered continuously (e.g., without a substantial quiescent period between packets or bursts of pulses).

In some embodiments, one or more parameters of the electrical signal can be ramped. For example, the amplitude of the electrical signal can be gradually and/or incrementally increased or decreased across one or more subsequent pulses or on-periods. For example, the amplitude of the signal may be gradually from about 90% of the perception threshold to about 150% of the motor threshold. Other examples include ramping the amplitude from about 70%, 80%, 90%, or 95% of the motor threshold to about 100%, 105%, 110%, 120%, 130%, 140%, 150%, 160%, 170%, 180%, 190%, or 200% of the motor threshold. In some embodiments, the ramping can occur over a period of between about 1 second to about 10 seconds. In some patients, ramping the signal can provide a smoother experience for the patient (e.g., starting with barely perceptible muscle twitching before increasing the strength of the motor contractions). In some embodiments, parameters other than amplitude (e.g., frequency, pulse width, pulse dosing, etc.) can be ramped.

Regardless of whether the electrical signal is continuous, non-continuous, and/or ramped, the electrical signal can be delivered during discrete stimulation sessions. The discrete stimulation sessions can last between about 10 minutes and 1 hour, or between about 15 minutes and 45 minutes, or between about 15 minutes and 30 minutes, or about 30 minutes. In such embodiments, stimulation (or at least stimulation that induces muscle contractions) is generally not administered between stimulation sessions. In some embodiments, the patient receives one, two, three, four, or more stimulation sessions per day. Additional details about delivering electrical stimulation according to stimulation sessions are described in U.S. Patent Publication No. 2024/0017068, the disclosure of which is incorporated herein by reference in its entirety. In embodiments in which the patient receives stimulation during discrete stimulation sessions, the signal generator can by preprogrammed to automatically initiate the stimulation sessions (e.g., based on an internal clock, measured physiological parameters, etc.). In other embodiments, the patient can control when the stimulation sessions occur (e.g., by initiating the sessions using a patient controller). In some embodiments, the stimulation sessions can be timed to coincide with periods when glucose levels are most likely to be elevated, such as after meals (postprandial hyperglycemia) or during periods of inactivity (fasting hyperglycemia).

In some embodiments, the method 400 can include monitoring a blood glucose level of the patient. For example, blood glucose can be measured before, during, and/or after delivering the electrical stimulation at block 404. In some embodiments, the electrical signal can be delivered in response to detecting elevated blood glucose levels. In some embodiments, a blood glucose level can be measured during and/or after delivering the electrical stimulation to assess the effectiveness of the treatment. A reduction in blood glucose levels compared to an initial measurement can indicate that the stimulation has successfully facilitated glucose uptake and lowered blood glucose levels.

Although the method 400 describes delivering electrical stimulation to a patient's spinal cord to induce motor contractions, in some embodiments the present technology include delivering electrical stimulation to other neural structures and targets. For example, in some embodiments, the present technology includes methods and systems for stimulating peripheral nerves, such as the sciatic nerve, tibial nerve, fibular nerve, and so forth, to induce muscle contractions in the legs. The sciatic nerve innervates, for example, the hamstrings, adductors, and muscles of the lower leg and foot. The muscle contractions increase the demand for glucose in the muscle cells, facilitating glucose uptake from the bloodstream via GLUT4 in an insulin-independent manner. Additionally, the tibial and fibular (peroneal) nerves, which are branches of the sciatic nerve, can be targeted to induce specific muscle contractions in the lower leg. For example, the tibial nerve innervates muscles such as the gastrocnemius, soleus, and flexor muscles of the foot, while the fibular nerve innervates muscles such as the tibialis anterior, extensor digitorum longus, and peroneal muscles. Implantable signal delivery devices (e.g., signal delivery device 110) can be positioned along the course of the nerves (e.g., near the popliteal fossa for the tibial nerve, near the fibular head for the fibular nerve) to electrically stimulation the nerves. In still further embodiments, electrical stimulation can be delivered to the patient's motor cortex.

### B. Maintaining Decreased Blood Glucose, Including Stimulation Schedules and Associated Methods

In some embodiments, electrical stimulation is delivered to the patient based on the patient's activity levels. For example, patient activity levels can be monitored using various sensors. If the patient activity level is below a certain threshold (e.g., suggesting the patient is in an inactive or sedentary state), then electrical stimulation can be delivered to the patient to induce muscle contractions and promote glucose uptake. If the patient activity level is above a certain threshold (e.g., suggesting the patient is in an active or non-sedentary state), then electrical stimulation is ceased or prevented from being delivered. In this way, electrical stimulation can be predominantly delivered during inactive or sedentary states. This may be advantageous for a number of reasons. First, delivering stimulation during times when the patient is sedentary or inactive may be less disruptive to the patient's lifestyle, e.g., by avoiding interfering with intentional patient movements. Second, delivering stimulation during times when the patient is sedentary or inactive induces muscle contraction, and thus glucose uptake, during periods that the patient likely would not otherwise have significant muscle contraction and thus significant insulin-independent glucose uptake, thus increasing the total amount of insulin-independent glucose uptake that occurs throughout the day.

FIG. 5 is a block diagram illustrating another method 500 of treating a patient in accordance with some embodiments of the present technology. Similar to the method 400 described with reference to FIG. 4, some or all of the operations in the method 500 shown in FIG. 5 can be performed by a processor executing instructions stored on one or more elements of a patient treatment system, including the patient treatment system 100 described with reference to FIG. 1.

The method 500 can begin at block 502 by delivering electrical stimulation to a target neural population within a spinal column of the patient and that is associated with motor control of a lower extremity or an abdomen of the patient. The electrical stimulation can be generally similar to or the same as the electrical signal described above in reference to FIG. 4. Thus, the electrical stimulation can have any of the parameters, settings, or features as described above with reference to FIG. 4.

The method 500 can continue at block 504 by determining whether the patient's activity is above a threshold value. The determination can be made using various sensors and monitoring devices that track the patient's physical activity levels. For instance, accelerometers and/or gyroscopes embedded in wearable devices or implanted sensors (e.g., within signal generator 101) can continuously or periodically monitor the patient's movements and provide real-time or near real-time data on the patient's activity levels. Accelerometers can measure a rate of change in velocity to detect movements such as walking, standing up, or shifting positions. Gyroscopes can measure the rate of rotation to determine the orientation and angular movement of the patient. The threshold value can be predefined based on clinical guidelines and/or patient-specific factors (e.g., age, weight, neural activity, blood glucose levels), or dynamically defined using historical patient data (e.g., historical blood glucose levels, historical baseline activity levels, etc.). In some embodiments, the patient can be periodically reevaluated to determine whether the threshold needs to be adjusted.

In some embodiments, if the patient's activity level exceeds the threshold, the system can classify the patient as being in an active state. Conversely, if the activity level is below the threshold, the patient can be considered to be in a sedentary state. The binary classification enables delivery of the electrical stimulation only when the patient is in a sedentary state to increase therapeutic efficacy and decrease unnecessary stimulation. In some embodiments, the threshold value can be based on the patient's heart rate, which can be monitored using a heart rate sensor. For instance, a threshold heart rate of 100 beats per minute (bpm) can be set. If the patient's heart rate exceeds this value, the patient is classified as being in an active state, and the electrical stimulation can be paused. If the heart rate is below this threshold, the patient is considered to be in a sedentary state, and the electrical stimulation can be resumed. In some embodiments, the threshold value can be patient-specific to account for patients with naturally low or high resting heart rates, and can range between, e.g., 70 bpm and 130 bpm. Additionally or alternatively, the threshold value can be determined using metabolic equivalents (METs), a standardized measure of the energy cost of physical activities. For example, a threshold MET value of 1.5 can be used to distinguish between sedentary and active states. If the patient's activity level exceeds the threshold, it indicates that the patient is engaged in physical activity. Conversely, if the MET value is below this threshold, the patient is considered to be in a sedentary state. METs can be calculated using various methods, including wearable devices that monitor heart rate, accelerometer data, and other physiological parameters.

In some embodiments, the threshold value can be determined using a combination of multiple parameters described above (e.g., heart rate, METs, historical patient data). For example, a composite threshold can be defined as a heart rate of 100 bpm, and a MET value of 1.5. If any of the parameters exceed their respective thresholds, the system can classify the patient as being in an active state. The patient can be considered to be in a sedentary state when their activity level is below a first threshold for a predefined period of time. For example, if the patient's activity level, measured in steps per minute or heart rate, remains below a threshold value such as 100 bpm for a continuous period of 5 minutes, the system can classify the patient as being in a sedentary state.

In some embodiments, the threshold value and/or delivery parameters of the electrical stimulation can be determined using one or more models (e.g., artificial intelligence models, machine learning models) that dynamically adjusts the threshold values based on the patient's historical activity data and response to the treatment. The model can evaluate trends and patterns in the patient's activity levels or blood glucose levels and adjust the threshold values, timing, and intensity of the electrical stimulation accordingly. For instance, if the model detects that the patient's blood glucose levels tend to spike after meals, the model can adjust the stimulation schedule to provide more frequent or intense stimulation during these particular periods. Similarly, if the model identifies that the patient is more active during certain times of the day, the model can adjust the threshold values to avoid unnecessary stimulation during these particular periods. Examples of the one or more machine learning models used are discussed further below under Heading 8.0 and elsewhere herein.

In some embodiments, the model is a machine learning model trained to identify specific patterns and correlations within the patient's activity data, blood glucose levels, and/or historical response to electrical stimulation. For example, the model can be trained on historical records of the patient's blood glucose levels at various times of the day, under different conditions, and in response to various activities and stimulation parameters. The model can use the training data to identify, for example, the impact of particular electrical stimulation parameters (e.g., packeted high-frequency stimulation, low frequency stimulation, etc.) on the patient's blood glucose levels. The model maps input features (e.g., activity levels, time of day, historical glucose readings) to output labels (e.g., effective stimulation parameters, effective threshold values). The model can dynamically adjust the threshold values and delivery parameters of the electrical stimulation in near real-time. For example, the model can receive incoming patient data to determine the patient's current activity level and blood glucose status. Further, the model can predict the suitable stimulation parameters, such as frequency, pulse width, and amplitude, and adjust the threshold values accordingly.

In some embodiments, the threshold value is determined using a rule-based engine within the signal generator 101. The rule-based engine can use a set of predefined rules and/or conditions to also determine, for example, stimulation parameters, stimulation schedules, and so forth. The predefined rules and/or conditions can be based on various objective and/or subjective criteria. Suitable objective criteria include, for example, age, weight, blood glucose levels, neural activity, patient activity level, etc. Suitable subjective criteria include, for example, patient-reported pain scores (e.g., VAS pain scores, NRS scores, etc.), patient-reported quality of life, patient satisfaction, patient sleep quality, physician observations, etc. For example, the engine can adjust the threshold value based on the patient's age, setting a lower threshold for older patients who may have reduced mobility and a higher threshold for younger, more active patients. Similarly, the engine can consider the patient's weight, adjusting the threshold value to account for the increased energy expenditure required for movement in heavier individuals. Additionally, the rule-based engine can use blood glucose levels to determine the threshold value. For instance, if the patient's blood glucose level is elevated, the engine can lower the activity threshold to ensure that electrical stimulation is delivered more frequently. Conversely, if the blood glucose level is within a “normal” range, the engine can raise the threshold value, reducing the frequency ′f stimulation and reducing unnecessary interventions.

If the patient's activity is determined as being above the threshold value at block 504, the method 500 can optionally continue at block 506 by automatically pausing the delivery of the electrical stimulation. The pausing mechanism ensures that the electrical stimulation is not delivered during periods of physical activity. For instance, if the patient's activity level exceeds the predefined threshold, such as a heart rate of 100 bpm, the system can cease electrical stimulation. Alternatively, if the patient's activity is determined as not being above the threshold value at block 504, the method 500 can optionally continue at block 508 by maintaining the delivery of the electrical stimulation. By maintaining the stimulation during these periods, the method can increase the therapeutic effect and lower blood glucose levels in patients with blood glucose-related conditions (e.g., NIDDM). Once the patient's activity level falls below the threshold again, the system can automatically restart the electrical stimulation. In some embodiments, the operations in blocks 502, 504, and 508 can be iteratively performed, e.g., until a daily maximum of stimulation is reached and/or a target therapeutic effect is achieved (e.g., a target blood glucose level).

Although the method 500 of FIG. 5 describes delivering the electrical stimulation and monitoring patient activity level, one skilled in the art will appreciate from the disclosure herein that the patient activity level can also be monitored while the patient is active. For example, patient activity level can be continuously or periodically monitored and, in response to the activity level falling below the threshold, stimulation can be initiated. In such embodiments, the stimulation can begin immediately upon the activity level crossing the threshold, or after some predefined buffer time (e.g., 2 minutes, 5 minutes, etc.) to ensure that the reduction in activity level is not merely transient. In some embodiments, the stimulation continues until the activity level exceeds the threshold. In other embodiments, once initiated, the stimulation proceeds for a predetermined duration (e.g., 15 minutes, 30 minutes, etc.).

As one skilled in the art will appreciate from the disclosure herein, the methods 400 and 500 described with respect to FIGS. 4 and 5 can be performed by other stimulation systems, including any variation of the system 100 described with respect to FIG. 1, and other stimulation systems known in the art.

### 5.0 Representative Stimulation Parameters

The electrical signals described herein, including the electrical signal described with respect to the method 400 and/or the electrical stimulation described with respect to method 500, can have a frequency in a frequency range of from about 0.1 Hz to about 100 kHz. For example, the electrical signal can have a frequency within a first (e.g., high) frequency band from about 500 Hz to about 100 kHz, or from about 500 Hz to 25 kHz, or from about 1 kHz to about 20 kHz, or from about 2 kHz to about 15 kHz, or from about 3 kHz to about 15 kHz, or from about 5 kHz to about 15 kHz, or about 10 kHz. Alternatively, the electrical signal can have a frequency within a second (e.g., low) frequency band of from about 0.1 Hz to about 10 Hz, or from about 0.1 Hz to about 2 Hz, or from about 0.1 Hz to about 1 Hz, or from about 0.5 Hz to about 2 Hz. Further yet, the electrical signal can have a frequency within a third (e.g., middle) frequency band of from about 10 Hz to about 50 Hz, or from about 20 Hz to about 50 Hz, or from about 30 Hz to about 50 Hz. In some embodiments, the electrical signals described herein can include multiple electrical signals being administered at different frequencies (e.g., a first, high frequency signal paired with a second, low frequency signal). These frequencies can be selected based on the patient's physiological responses (e.g., the perception threshold and the motor threshold of the patient described with respect to method 400 and method 500).

The electrical signal may have a pulse width of from about 10 microseconds to about 1 second. For example, the electrical signal may have a pulse width of from about 10 microseconds to about 417 microseconds, or from about 10 microseconds to about 333 microseconds, or from about 10 microseconds to about 166 microseconds, or from about 25 microseconds to about 166 microseconds, or from about 20 microseconds to about 100 microseconds, or from about 30 microseconds to about 100 microseconds, or from about 30 microseconds to about 40 microseconds, or from about 10 microseconds to about 50 microseconds, or from about 20 microseconds to about 40 microseconds, or from about 25 microseconds to about 35 microseconds, or from about 30 microseconds to about 35 microseconds, or 30 microseconds. As additional examples, the electrical signal may have a pulse width of from about 1 milliseconds to about 1 second, or between about 1 millisecond and about 500 milliseconds, or between about 100 milliseconds and about 500 milliseconds, In some embodiments, the electrical signal can be administered at current amplitudes of from 0.1 mA to 20 mA, or 0.5 mA to 10 mA, or 0.5 mA to 7 mA, or 0.5 mA to 5 mA. The electrical signal can also be administered according to a pulse dose rate ranging from about 1% to about 100%, such as about 1% to about 50%, or about 2% to about 20%, or about 3% to about 15%, or about 3%, about 6%, about 9%, about 14%, or about 20%. Without intending to be bound by theory, the intermittent stimulation can still achieve therapeutic effects in certain patients at a lower energy consumption.

In some embodiments, one or more of the parameters of the electrical signal may be “ramped,” or gradually and/or incrementally increased or decreased in value. For example, an amplitude of the electrical signal can be ramped from 90% of the perception threshold to 150% of the motor threshold, from 80% of the perception threshold to 120% of the motor threshold, from 85% of the perception threshold to 130% of the motor threshold, from 95% of the perception threshold to 140% of the motor threshold, from 100% of the perception threshold to 160% of the motor threshold, or from 90% of the perception threshold to 170% of the motor threshold. Other examples include ramping the amplitude from about 70%, 80%, 90%, or 95% of the motor threshold to about 100%, 105%, 110%, 120%, or 130% of the motor threshold. The ramping can occur over various time periods, such as from about 1 second to about 10 seconds, or from about 2 seconds to about 5 seconds.

In some embodiments, the electrical signal can be delivered in bursts or packets according to a pulse dosing regimen. Example pulse dosing regimens include periods of 1-10 seconds “on” and 1-10 seconds “off”, 2-8 seconds “on” and 2-8 seconds “off”, 3-7 seconds “on” and 3-7 seconds “off”, 4-6 seconds “on” and 4-6 seconds “off”, 5-10 seconds “on” and 5-10 seconds “off”, or 1-5 seconds “on” and 1-5 seconds “off”. In other embodiments, the electrical signal is continuous or substantially continuous.

The electrical signal may be delivered according to scheduled periods of stimulation (e.g., discrete stimulation sessions). The discrete stimulation sessions can last between about 10 minutes and 1 hour, or between about 15 minutes and 45 minutes, or between about 15 minutes and 30 minutes, or about 30 minutes. In such embodiments, stimulation (or at least stimulation that induces muscle contractions) is generally not administered between stimulation sessions. In some embodiments, the patient receives one, two, three, four, or more stimulation sessions per day. Additional details about delivering electrical stimulation according to stimulation sessions are described in U.S. Patent Publication No. 2024/0017068, the disclosure of which is incorporated herein by reference in its entirety.

In some embodiments, the electrical signals do not produce paresthesia when delivered to the patient, and can therefore be referred to as “non-paresthesia producing electrical signals,” or “paresthesia-free signals. ” As set forth previously, such signals have combinations of frequency, pulse widths, amplitudes, and/or pulse dose rates that cause the signal to be below a patient's perception threshold. For example, some paresthesia-free electrical signals may have a frequency of between about 0.01 Hz and about 1 MHz, or between about 1.2 kHz and 500 kHz, or between about 1.2 kHz and about 100 kHz. As another example, some paresthesia-free electrical signals may be delivered in discrete bursts, separated by quiescent periods in which the electrical signal is not delivered. As yet another example, some paresthesia-free electrical signals may have a “long” pulse width of between about 5 milliseconds and about 25 seconds with an amplitude below an activation threshold of a target neural population. Additional examples of paresthesia-free electrical signals, including those mentioned above, are described in U.S. Patent Application Publication Nos. US2010/0274314, US2021/0228881, and US2022/0401730, and International Patent Application No. PCT/US2024/028654, each of the which is incorporated by reference herein.

In some embodiments, the electrical signals described herein may produce paresthesia when delivered to the patient, and can therefore be referred to as “paresthesia-producing” signals. Such signals have combinations of frequency, pulse widths, amplitudes, and/or pulse dosing rates that cause the signal to be above a patient's perception threshold.

Any of the foregoing signal parameters can be combined. For example, electrical signals having any of the described frequencies, pulse widths, and amplitude values can be ramped, delivered according to a pulse dosing regimen, and/or delivered during discrete stimulation sessions.

### 6.0 Representative Clinical Applications

The present technology has generally been described in the context of treating hyperglycemia. For example, stimulation delivered at or above the motor threshold of the patient increases the patient's glucose uptake and treats, reduces, and/or prevents hyperglycemia. Without being bound by theory, it is expected that many different types of hyperglycemia can be treated using the electrical stimulation described herein. For example, the electrical stimulation described herein can be applied to treat hyperglycemia associated with various conditions, including NIDDM, gestational diabetes, and/or prediabetes, among others. Similarly, the electrical stimulation described herein can be applied to treat hyperglycemia resulting from various causes, such as insulin resistance, pancreatic dysfunction, and/or medication-induced hyperglycemia, among others. The electrical stimulation described herein can also be applied to treat hyperglycemia in different patient populations, including adults, children, and/or pregnant individuals, among others. Additionally, the electrical stimulation can be tailored to address hyperglycemia occurring at different times of the day, such as fasting hyperglycemia, postprandial hyperglycemia, and/or nocturnal hyperglycemia, among others. The stimulation schedules described herein can also be applied to manage hyperglycemia in patients with comorbid conditions, such as obesity, hypertension, and dyslipidemia.

The electrical stimulation described herein can also be used to treat indications or symptoms other than hyperglycemia. For example, the electrical stimulation described herein can be used to treat insulin resistance or metabolic dysfunctions (e.g., insulin resistance associated with metabolic syndrome or other metabolic disorders). In such embodiments, stimulation delivered during the stimulation period improves insulin sensitivity/reduces insulin resistance. The stimulation schedules described herein can also be used to treat abnormal glucose metabolism, such as impaired glucose tolerance or prediabetes (e.g., conditions that precede the development of NIDDM). Of course, the stimulation schedules described herein may be applied to treat indications and/or symptoms beyond those expressly recited herein.

### 7.0 Pre-Clinical Data

Nevro Corp., the applicant of the present application, has conducted animal studies of certain HbA1c level management methods described herein. One study included cohorts of both “diabetic models” (e.g., rats that had been injected with streptozotocin (STZ) to induce diabetes, and thus were exhibiting elevated blood glucose levels and impaired glucose uptake) and “naive/healthy models” (e.g., rats that had not been injected with STZ and thus had normal glucose metabolism). The diabetic models and the naive models were assigned to different testing protocols: (1) the diabetic models were tested at 2 weeks post-STZ injection, and (2) the naive models were tested without any prior intervention. Both cohorts underwent glucose tolerance testing (GTT), in which they were injected with 2 g/kg glucose in a saline solution intraperitoneally. Blood glucose levels were then measured from the tail vein at regular intervals for the next 90-120 minutes post-injection.

FIG. 6 is a graph 600 showing the changes of blood glucose levels of both the diabetic rats and naïve rats following the intraperitoneal injection, in accordance with embodiments of the present technology. In particular, graph 600 illustrates the recorded blood glucose levels for a first naive rat (line 602), recorded blood glucose levels for a second naive rat (line 604), recorded blood glucose levels for a first diabetic rat (line 606), and recorded blood glucose levels for a second diabetic rat (line 608) at various intervals in time. The x-axis measures the time in minutes, and the y-axis measure the blood glucose level in milligrams per deciliter (mg/dl). The data in graph 600 shows the rats response to the intraperitoneal injection of glucose in the absence of receiving any electrical stimulation. As shown on the x-axis, blood glucose levels were measured at baseline, at 15 minutes, at 30 minutes, at 60 minutes, and at 120 minutes. Baseline refers to the blood glucose level before the glucose injection. The 30-minute measurement represents the glucose level peak for naive animals, which generally show a normal, “low-level” glucose spike. The 60-minute, 90-minute, and 120-minute measurements represent the blood glucose levels as they return to baseline or remain elevated. As shown in graph 600, the naïve rats exhibit a normal glucose spike peaking at 30 minutes and then returning to baseline levels. In contrast, the diabetic rats show elevated baseline glucose levels and slow or no glucose uptake from 90 to 120 minutes post-injection, indicating impaired glucose metabolism.

FIG. 7 is a graph 700 showing the changes of blood glucose levels of a particular diabetic rat following the intraperitoneal injection of glucose but while receiving spinal column stimulation above the motor threshold of the particular diabetic rat, in accordance with embodiments of the present technology. In particular, graph 700 illustrates recorded blood glucose levels (line 702), the time at which glucose is injected (704) for the particular diabetic rat, and the period of SCS delivery (706). The x-axis measures the time in minutes, and the y-axis measure the blood glucose level in mg/dl. The particular diabetic rat received 15 minutes of SCS at 124% of the motor threshold. A rat was considered responsive if the rat had a significant reduction in blood glucose levels relative to their baseline glucose level within the 15-minute stimulation period, despite receiving a glucose injection. For the particular diabetic rat, the baseline glucose level was the rat's glucose level before receiving any SCS therapy/electrical stimulation. As shown in FIG. 7, the particular diabetic rat experienced a substantial reduction in blood glucose levels during the 15 minutes of SCS at 124% of the motor threshold (e.g., from about 500 mg/dl to about 370 mg/dl). Unlike the elevation of blood glucose levels observed after glucose injection in FIG. 6, the electrical stimulation delivered to the rat in FIG. 7 facilitated the extraction of excess blood glucose from the bloodstream of the rat. Following the cessation of stimulation, the blood glucose levels rose again due to the inherent insulin resistance of the tissues and relative hyperglycemia.

Taken together, the data in FIGS. 6 and 7 support that, at least for some patients, SCS at or above the motor threshold can provide effective therapy for managing blood glucose levels. For example, the study demonstrates that naive rats show a normal, low-level glucose spike, peaking at 30 minutes, while diabetic rats show elevated baseline glucose levels and slow or no glucose uptake from 90 to 120 minutes post-injection. However, the particular diabetic rat, which received 15 minutes of SCS at 124% of the motor threshold timed with the glucose injection, exhibited a reduction in blood glucose levels during the stimulation period. Although data is only reported for this particular stimulation schedule (i.e., 15 minutes of SCS at 124% of the motor threshold), it is expected that other SCS schedules described herein will similarly provide effective therapy.

### 8.0 Representative Systems Used to Determine Stimulation Parameters

### A. AI System

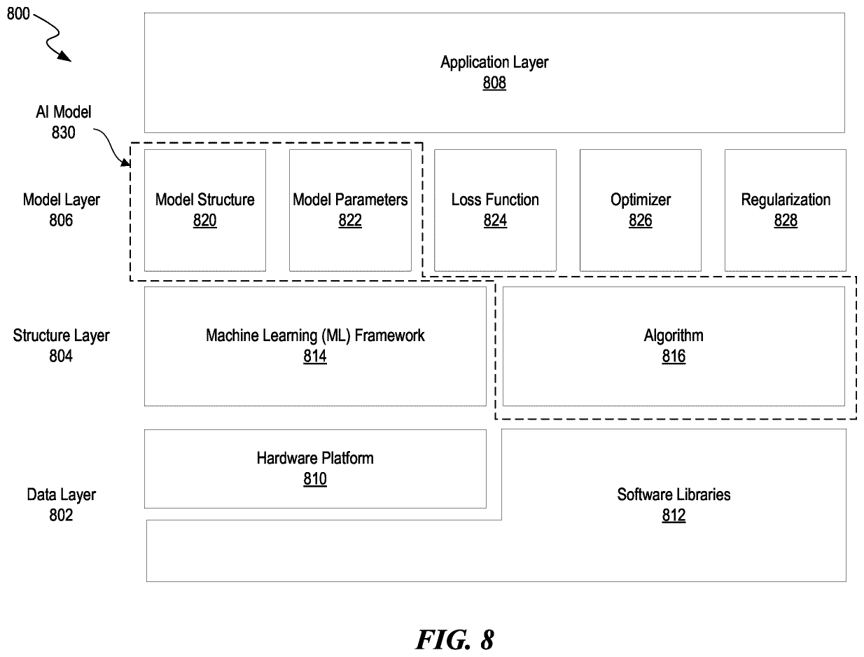

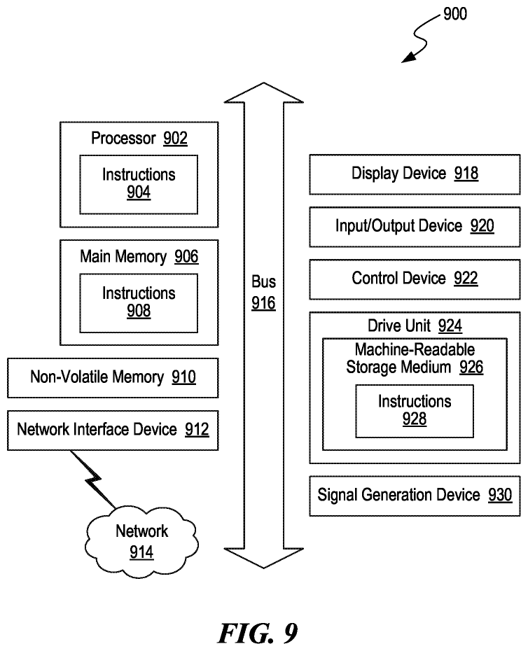

FIG. 8 is a block diagram illustrating an example artificial intelligence (AI) system 800, in accordance with one or more implementations of this disclosure. The AI system 800 is implemented using components of the example computer system 900 illustrated and described in more detail with reference to FIG. 9. For example, the AI system 800 can be implemented using the processor 902 and instructions 908 programmed in the memory 906 illustrated and described in more detail with reference to FIG. 9. Likewise, implementations of the AI system 800 can include different and/or additional components or be connected in different ways.

As shown, the AI system 800 can include a set of layers, which conceptually organize elements within an example network topology for the AI system's architecture to implement a particular AI model 830. Generally, an AI model 830 is a computer-executable program implemented by the AI system 800 that analyzes data to make predictions. Information can pass through each layer of the AI system 800 to generate outputs for the AI model 830. The layers can include a data layer 802, a structure layer 804, a model layer 806, and an application layer 808. The algorithm 816 of the structure layer 804 and the model structure 820 and model parameters 822 of the model layer 806 together form the example AI model 830. The optimizer 826, loss function engine 824, and regularization engine 828 work to refine and optimize the AI model 830, and the data layer 802 provides resources and support for application of the AI model 830 by the application layer 808.

The data layer 802 acts as the foundation of the AI system 800 by preparing data for the AI model 830. As shown, the data layer 802 can include two sub-layers: a hardware platform 810 and one or more software libraries 812. The hardware platform 810 can be designed to perform operations for the AI model 830 and include computing resources for storage, memory, logic, and networking, such as the resources described in relation to FIG. 9. The hardware platform 810 can process amounts of data using one or more servers. The servers can perform backend operations such as matrix calculations, parallel calculations, machine learning (ML) training, and the like. Examples of servers used by the hardware platform 810 include central processing units (CPUs) and graphics processing units (GPUs). CPUs are electronic circuitry designed to execute instructions for computer programs, such as arithmetic, logic, controlling, and input/output (I/O) operations, and can be implemented on integrated circuit (IC) microprocessors. GPUs are electric circuits that were originally designed for graphics manipulation and output but can be used for AI applications due to their vast computing and memory resources. GPUs use a parallel structure that generally makes their processing more efficient than that of CPUs. In some instances, the hardware platform 810 can include Infrastructure as a Service (IaaS) resources, which are computing resources, (e.g., servers, memory, etc.) offered by a cloud services provider. The hardware platform 810 can also include computer memory for storing data about the AI model 830, application of the AI model 830, and training data for the AI model 830. The computer memory can be a form of random-access memory (RAM), such as dynamic RAM, static RAM, and non-volatile RAM.

The software libraries 812 can be thought of as suites of data and programming code, including executables, used to control the computing resources of the hardware platform 810. The programming code can include low-level primitives (e.g., fundamental language elements) that form the foundation of one or more low-level programming languages, such that servers of the hardware platform 810 can use the low-level primitives to carry out specific operations. The low-level programming languages do not require much, if any, abstraction from a computing resource's instruction set architecture, allowing them to run quickly with a small memory footprint. Examples of software libraries 812 that can be included in the AI system 800 include INTEL MATH KERNEL LIBRARY, NVIDIA CUDNN, EIGEN, AND OPEN BLAS.

The structure layer 804 can include a machine learning (ML) framework 814 and an algorithm 816. The ML framework 814 can be thought of as an interface, library, or tool that allows users to build and deploy the AI model 830. The ML framework 814 can include an open-source library, an application programming interface (API), a gradient-boosting library, an ensemble method, and/or a deep learning toolkit that work with the layers of the AI system facilitate development of the AI model 830. For example, the ML framework 814 can distribute processes for application or training of the AI model 830 across multiple resources in the hardware platform 810. The ML framework 814 can also include a set of pre-built components that have the functionality to implement and train the AI model 830 and allow users to use pre-built functions and classes to construct and train the AI model 830. Thus, the ML framework 814 can be used to facilitate data engineering, development, hyperparameter tuning, testing, and training for the AI model 830.

Examples of ML frameworks 814 or libraries that can be used in the AI system 800 include TENSORFLOW, PYTORCH, SCIKIT-LEARN, KERAS, and CAFFFE. Random Forest is a machine learning algorithm that can be used within the ML frameworks 814. LightGBM is a gradient boosting framework/algorithm (an ML technique) that can be used. Other techniques/algorithms that can be used are XGBoost, CatBoost, etc. AMAZON WEB SERVICES is a cloud service provider that offers various machine learning services and tools (e.g., SAGE MAKER) that can be used for platform building, training, and deploying ML models.

In some implementations, the ML framework 814 performs deep learning (also known as deep structured learning or hierarchical learning) directly on the input data to learn data representations, as opposed to using task-specific algorithms. In deep learning, no explicit feature extraction is performed; the features of feature vector are implicitly extracted by the AI system 800. For example, the ML framework 814 can use a cascade of multiple layers of nonlinear processing units for implicit feature extraction and transformation. Each successive layer uses the output from the previous layer as input. The AI model 830 can thus learn in supervised (e.g., classification) and/or unsupervised (e.g., pattern analysis) modes. The AI model 830 can learn multiple levels of representations that correspond to different levels of abstraction, wherein the different levels form a hierarchy of concepts. In this manner, AI model 830 can be configured to differentiate features of interest from background features.

The algorithm 816 can be an organized set of computer-executable operations used to generate output data from a set of input data and can be described using pseudocode. The algorithm 816 can include complex code that allows the computing resources to learn from new input data and create new/modified outputs based on what was learned. In some implementations, the algorithm 816 can build the AI model 830 through being trained while running computing resources of the hardware platform 810. This training allows the algorithm 816 to make predictions or decisions without being explicitly programmed to do so. Once trained, the algorithm 816 can run at the computing resources as part of the AI model 830 to make predictions or decisions, improve computing resource performance, or perform tasks. The algorithm 816 can be trained using supervised learning, unsupervised learning, semi-supervised learning, and/or reinforcement learning.

Using supervised learning, the algorithm 816 can be trained to learn patterns (e.g., map input data to output data) based on labeled training data. The training data can be labeled by an external user or operator. For instance, a user can collect a set of training data, such as by capturing patient blood glucose levels, historical stimulation parameters, historical threshold values, and the like (detailed further above, under Heading 4.0 and elsewhere herein). The user can label the training data based on one or more classes and trains the AI model 830 by inputting the training data to the algorithm 816. The algorithm determines how to label the new data based on the labeled training data. The user can facilitate collection, labeling, and/or input via the ML framework 814. In some instances, the user can convert the training data to a set of feature vectors for input to the algorithm 816. Once trained, the user can test the algorithm 816 on new data to determine if the algorithm 816 is predicting accurate labels for the new data. For example, the user can use cross-validation methods to test the accuracy of the algorithm 816 and retrain the algorithm 816 on new training data if the results of the cross-validation are below an accuracy threshold.

Supervised learning can involve classification and/or regression. Classification techniques involve teaching the algorithm 816 to identify a category of new observations based on training data and are used when input data for the algorithm 816 is discrete. Said differently, when learning through classification techniques, the algorithm 816 receives training data labeled with categories (e.g., classes) and determines how features observed in the training data (e.g., features of patient data, under Heading 4.0 and elsewhere herein) relate to the categories (e.g., active or sedentary). Once trained, the algorithm 816 can categorize new data by analyzing the new data for features that map to the categories. Examples of classification techniques include boosting, decision tree learning, genetic programming, learning vector quantization, k-nearest neighbor (k-NN) algorithm, and statistical classification.

Regression techniques involve estimating relationships between independent and dependent variables and are used when input data to the algorithm 816 is continuous. Regression techniques can be used to train the algorithm 816 to predict or forecast relationships between variables. To train the algorithm 816 using regression techniques, a user can select a regression method for estimating the parameters of the model. The user collects and labels training data that is input to the algorithm 816 such that the algorithm 816 is trained to understand the relationship between data features and the dependent variable(s). Once trained, the algorithm 816 can predict missing historic data or future outcomes based on input data. Examples of regression methods include linear regression, multiple linear regression, logistic regression, regression tree analysis, least squares method, and gradient descent. In an example implementation, regression techniques can be used, for example, to estimate and fill-in missing data for machine-learning based pre-processing operations.

Under unsupervised learning, the algorithm 816 learns patterns from unlabeled training data. In particular, the algorithm 816 is trained to learn hidden patterns and insights of input data, which can be used for data exploration or for generating new data. Here, the algorithm 816 does not have a predefined output, unlike the labels output when the algorithm 816 is trained using supervised learning. Another way unsupervised learning is used to train the algorithm 816 to find an underlying structure of a set of data is to group the data according to similarities and represent that set of data in a compressed format. The network 100 disclosed herein can use unsupervised learning to identify patterns in data received.

A few techniques can be used in supervised learning: clustering, anomaly detection, and techniques for learning latent variable models. Clustering techniques involve grouping data into different clusters that include similar data, such that other clusters contain dissimilar data. For example, during clustering, data with possible similarities remain in a group that has less or no similarities to another group. Examples of clustering techniques density-based methods, hierarchical based methods, partitioning methods, and grid-based methods. In one example, the algorithm 816 can be trained to be a k-means clustering algorithm, which partitions n observations in k clusters such that each observation belongs to the cluster with the nearest mean serving as a prototype of the cluster. Anomaly detection techniques are used to detect previously unseen rare objects or events represented in data without prior knowledge of these objects or events. Anomalies can include data that occur rarely in a set, a deviation from other observations, outliers that are inconsistent with the rest of the data, patterns that do not conform to well-defined normal behavior, and the like. When using anomaly detection techniques, the algorithm 816 can be trained to be an Isolation Forest, local outlier factor (LOF) algorithm, or K-nearest neighbor (k-NN) algorithm. Latent variable techniques involve relating observable variables to a set of latent variables. These techniques assume that the observable variables are the result of an individual's position on the latent variables and that the observable variables have nothing in common after controlling for the latent variables. Examples of latent variable techniques that can be used by the algorithm 816 include factor analysis, item response theory, latent profile analysis, and latent class analysis.

In some implementations, the AI system 800 trains the algorithm 816 of AI model 830, based on the training data, to correlate the feature vector to expected outputs in the training data. As part of the training of the AI model 830, the AI system 800 forms a training set of features and training labels by identifying a positive training set of features that have been determined to have a desired property in question, and, in some implementations, forms a negative training set of features that lack the property in question. The AI system 800 applies ML framework 814 to train the AI model 830, that when applied to the feature vector, outputs indications of whether the feature vector has an associated desired property or properties, such as a probability that the feature vector has a particular Boolean property, or an estimated value of a scalar property. The AI system 800 can further apply dimensionality reduction (e.g., via linear discriminant analysis (LDA), PCA, or the like) to reduce the amount of data in the feature vector to a smaller, more representative set of data.

The model layer 806 implements the AI model 830 using data from the data layer and the algorithm 816 and ML framework 814 from the structure layer 804, thus enabling decision-making capabilities of the AI system 800. The model layer 806 includes a model structure 820, model parameters 822, a loss function engine 824, an optimizer 826, and a regularization engine 828.

The model structure 820 describes the architecture of the AI model 830 of the AI system 800. The model structure 820 defines the complexity of the pattern/relationship that the AI model 830 expresses. Examples of structures that can be used as the model structure 820 include decision trees, support vector machines, regression analyses, Bayesian networks, Gaussian processes, genetic algorithms, and artificial neural networks (or, simply, neural networks). The model structure 820 can include a number of structure layers, a number of nodes (or neurons) at each structure layer, and activation functions of each node. Each node's activation function defines how to node converts data received to data output. The structure layers can include an input layer of nodes that receive input data, an output layer of nodes that produce output data. The model structure 820 can include one or more hidden layers of nodes between the input and output layers. The model structure 820 can be an Artificial Neural Network (or, simply, neural network) that connects the nodes in the structured layers such that the nodes are interconnected. Examples of neural networks include Feedforward Neural Networks, convolutional neural networks (CNNs), Recurrent Neural Networks (RNNs), Autoencoder, and Generative Adversarial Networks (GANs).

The model parameters 822 represent the relationships learned during training and can be used to make predictions and decisions based on input data. The model parameters 822 can weight and bias the nodes and connections of the model structure 820. For instance, when the model structure 820 is a neural network, the model parameters 822 can weight and bias the nodes in each layer of the neural networks, such that the weights determine the strength of the nodes and the biases determine the thresholds for the activation functions of each node. The model parameters 822, in conjunction with the activation functions of the nodes, determine how input data is transformed into desired outputs. The model parameters 822 can be determined and/or altered during training of the algorithm 816.

The loss function engine 824 can determine a loss function, which is a metric used to evaluate the AI model's 830 performance during training. For instance, the loss function engine 824 can measure the difference between a predicted output of the AI model 830 and the actual output of the AI model 830 and is used to guide optimization of the AI model 830 during training to minimize the loss function. The loss function can be presented via the ML framework 814, such that a user can determine whether to retrain or otherwise alter the algorithm 816 if the loss function is over a threshold. In some instances, the algorithm 816 can be retrained automatically if the loss function is over the threshold. Examples of loss functions include a binary-cross entropy function, hinge loss function, regression loss function (e.g., mean square error, quadratic loss, etc.), mean absolute error function, smooth mean absolute error function, log-cosh loss function, and quantile loss function.

The optimizer 826 adjusts the model parameters 822 to minimize the loss function during training of the algorithm 816. In other words, the optimizer 826 uses the loss function generated by the loss function engine 824 as a guide to determine what model parameters lead to the most accurate AI model 830. Examples of optimizers include Gradient Descent (GD), Adaptive Gradient Algorithm (AdaGrad), Adaptive Moment Estimation (Adam), Root Mean Square Propagation (RMSprop), Radial Base Function (RBF) and Limited-memory BFGS (L-BFGS). The type of optimizer 826 used can be determined based on the type of model structure 820 and the size of data and the computing resources available in the data layer 802.

The regularization engine 828 executes regularization operations. Regularization is a technique that prevents over-and under-fitting of the AI model 830. Overfitting occurs when the algorithm 816 is overly complex and too adapted to the training data, which can result in poor performance of the AI model 830. Underfitting occurs when the algorithm 816 is unable to recognize even basic patterns from the training data such that it cannot perform well on training data or on validation data. The regularization engine 828 can apply one or more regularization techniques to fit the algorithm 816 to the training data properly, which helps constraint the resulting AI model 830 and improves its ability for generalized application. Examples of regularization techniques include lasso (L1) regularization, ridge (L2) regularization, and elastic (L1 and L2 regularization).

In some implementations, the AI system 800 can include a feature extraction module implemented using components of the example computer system 900 illustrated and described in more detail with reference to FIG. 9. In some implementations, the feature extraction module extracts a feature vector from input data. The feature vector includes n features (e.g., feature a, feature b,. feature n). The feature extraction module reduces the redundancy in the input data, e.g., repetitive data values, to transform the input data into the reduced set of features such as feature vector. The feature vector contains the relevant information from the input data, such that events or data value thresholds of interest can be identified by the AI model 830 by using this reduced representation. In some example implementations, the following dimensionality reduction techniques are used by the feature extraction module: independent component analysis, Isomap, kernel principal component analysis (PCA), latent semantic analysis, partial least squares, PCA, multifactor dimensionality reduction, nonlinear dimensionality reduction, multilinear PCA, multilinear subspace learning, semidefinite embedding, autoencoder, and deep feature synthesis.

### B. Computer System

FIG. 9 is a block diagram that illustrates an example of a computer system 900 in which at least some operations described herein can be implemented. As shown, the computer system 900 can include: one or more processors 902, main memory 906, non-volatile memory 910, a network interface device 912, a video display device 918, an input/output device 920, a control device 922 (e.g., keyboard and pointing device), a drive unit 924 that includes a machine-readable (storage) medium 926, and a signal generation device 930 that are communicatively connected to a bus 916. The bus 916 represents one or more physical buses and/or point-to-point connections that are connected by appropriate bridges, adapters, or controllers. Various common components (e.g., cache memory) are omitted from FIG. 9 for brevity. Instead, the computer system 900 is intended to illustrate a hardware device on which components illustrated or described relative to the examples of the figures and any other components described in this specification can be implemented.

The computer system 900 can take any suitable physical form. For example, the computing system 900 can share a similar architecture as that of a server computer, personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computing system 900. In some implementations, the computer system 900 can be an embedded computer system, a system-on-chip (SOC), a single-board computer system (SBC), or a distributed system such as a mesh of computer systems, or it can include one or more cloud components in one or more networks. Where appropriate, one or more computer systems 900 can perform operations in real time, in near real time, or in batch mode.

The network interface device 912 enables the computing system 900 to mediate data in a network 914 with an entity that is external to the computing system 900 through any communication protocol supported by the computing system 900 and the external entity. Examples of the network interface device 912 include a network adapter card, a wireless network interface card, a router, an access point, a wireless router, a switch, a multilayer switch, a protocol converter, a gateway, a bridge, a bridge router, a hub, a digital media receiver, and/or a repeater, as well as all wireless elements noted herein.

The memory (e.g., main memory 906, non-volatile memory 910, machine-readable medium 926) can be local, remote, or distributed. Although shown as a single medium, the machine-readable medium 926 can include multiple media (e.g., a centralized/distributed database and/or associated caches and servers) that store one or more sets of instructions 928. The machine-readable medium 926 can include any medium that is capable of storing, encoding, or carrying a set of instructions for execution by the computing system 900. The machine-readable medium 926 can be non-transitory or comprise a non-transitory device. In this context, a non-transitory storage medium can include a device that is tangible, meaning that the device has a concrete physical form, although the device can change its physical state. Thus, for example, non-transitory refers to a device remaining tangible despite this change in state.

Although implementations have been described in the context of fully functioning computing devices, the various examples are capable of being distributed as a program product in a variety of forms. Examples of machine-readable storage media, machine-readable media, or computer-readable media include recordable-type media such as volatile and non-volatile memory 910, removable flash memory, hard disk drives, optical disks, and transmission-type media such as digital and analog communication links.

In general, the routines executed to implement examples herein can be implemented as part of an operating system or a specific application, component, program, object, module, or sequence of instructions (collectively referred to as “computer programs”). The computer programs typically comprise one or more instructions (e.g., instructions 904, 908, 928) set at various times in various memory and storage devices in computing device(s). When read and executed by the processor 902, the instruction(s) cause the computing system 900 to perform operations to execute elements involving the various aspects of the disclosure.

### 9.0 Representative Examples

The following examples are provided to further illustrate embodiments of the present technology and are not to be interpreted as limiting the scope of the present technology. To the extent that certain embodiments or features thereof are mentioned, it is merely for purposes of illustration and, unless otherwise specified, is not intended to limit the present technology. It will be understood that many variations can be made in the procedures described herein while still remaining within the bounds of the present technology. Such variations are intended to be included within the scope of the presently disclosed technology.
1. A method for lowering blood glucose levels in a patient using spinal column stimulation, the method comprising:
programming a signal generator to:
   generate an electrical signal having an amplitude at or above a motor threshold of the patient, and
   deliver the electrical signal to a spinal column of the patient via an implanted signal delivery device positioned proximate a target neural population associated with the patient's abdomen and/or lower extremities,
wherein the electrical signal induces motor contractions in the patient's abdomen and/or lower extremities to reduce a blood glucose level in the patient.
2. The method of example 1, wherein the electrical signal has a frequency within a frequency range of from about 0.1 Hz to about 2 Hz.
3. The method of example 1, wherein the electrical signal has a frequency range of from about 500 Hz to about 10 kHz.
4. The method of example 1, wherein the electrical signal has a frequency range of from about 10 Hz to about 50 Hz.
5. The method of any of examples 1-4, wherein the electrical signal is delivered in periods of from about 1 second to about 10 seconds followed by a quiescent period of from about 1 second to about 10 seconds.
6. The method of any of examples 1-5, wherein an amplitude of the electrical signal is ramped from below the perception threshold to between about 100-150% of the motor threshold of the patient.
7. The method of example 6, wherein the electrical signal is ramped from about 90% of the perception threshold to about 150% of the motor threshold of the patient.
8. The method of any of examples 1-7, wherein programming the signal generator to generate and deliver the electrical signal includes programming the signal generator to generate and deliver the electrical signal during discrete stimulation sessions.
9. The method of example 8, wherein the discrete stimulation sessions include two or more stimulation sessions per day.

10. The method of example 8 or example 9, wherein each stimulation session has a duration of between about 15 minutes and about 45 minutes.

11. The method of any of examples 1-10, wherein programming the signal generator to generate and deliver the electrical signal includes programming the signal generator to:
receive an indication of an activity level of a patient;
in response to the activity level of the patient being below a predetermined threshold, generate and deliver the electrical signal, and
in response to the activity level of the patient exceeding the predetermined threshold, ceasing generating and delivering the electrical signal.
12. The method of example 1, wherein programming the signal generator is performed in response to the patient having an elevated blood glucose level.
13. The method of any of examples 1-12, wherein the electrical signal reduces the blood glucose level in the patient in an insulin-independent manner.
14. A method of decreasing blood glucose levels in a patient, the method comprising:
delivering electrical stimulation to a target neural population within a spinal column of the patient, wherein the target neural population is associated with motor control of a lower extremity or an abdomen of the patient, and wherein the electrical stimulation has an amplitude at or above a motor threshold,
wherein the electrical stimulation induces motor contractions in the lower extremity and/or abdomen of the patient to uptake glucose from the bloodstream of the patient.
15. The method of example 14, further comprising:
measuring a blood glucose level of the patient;
comparing the measured blood glucose level of the patient to a baseline glucose level of the patient; and
adjusting one or more signal parameters of the electrical stimulation based on the comparison.
16. The method of example 14, further comprising:
monitoring an activity level of the patient;
in response to the activity level of the patient exceeding a predetermined threshold indicating the patient is in an active state, ceasing deliver of the electrical stimulation; and
in response to the activity level returning to below the predetermined threshold, resuming deliver of the electrical stimulation.
17. The method of any of examples 14-16, further comprising:
determining the motor threshold of the patient;
delivering the electrical stimulation at an amplitude between about 100% and 150% of the motor threshold; and
adjusting the amplitude based on at least in part on the motor contractions.
18. The method of any of example 16, wherein the motor contractions are detected using electromyography (EMG) sensors placed on the lower extremity or the abdomen of the patient.
19. The method of any of examples 14-17, wherein the electrical stimulation is delivered through electrodes implanted in an epidural space of the spinal column of the patient.
20. A patient treatment system for lowering blood glucose levels in a patient using spinal column stimulation, comprising:
an implantable signal delivery device positionable proximate a target neural population within the patient's spinal column that is associated with the patient's abdomen and/or lower extremities; and
a signal generator having a controller programmed with instructions that, when executed, cause the signal generator to:
   deliver an electrical signal to the target neural population via the implantable signal delivery device,
   wherein the electrical signal has an amplitude at or above a motor threshold of the patient, and
   wherein the electrical signal induces motor contractions in the patient's abdomen and/or lower extremities to reduce a blood glucose level in the patient.
21. The system of example 20, wherein the electrical signal has a frequency within a frequency range of from about 0.1 Hz to about 2 Hz.
22. The system of example 20, wherein the electrical signal has a frequency range of from about 500 Hz to about 10 kHz.
23. The system of example 20, wherein the electrical signal has a frequency range of from about 10 Hz to about 50 Hz.
24. The system of any of examples 20-23, wherein the electrical signal is delivered in periods of from about 1 second to about 10 seconds followed by a quiescent period of from about 1 second to about 10 seconds.
25. The system of any of examples 20-24, wherein the amplitude of the electrical signal is ramped from below a perception threshold of the patient to the amplitude at or above the motor threshold.
26. The system of any of examples 20-25 wherein the implantable signal delivery device is positionable within an epidural space of the spinal column region of the patient.
27. The system of any of examples 20-26, wherein the electrical signal reduces the blood glucose level in the patient in an insulin-independent manner.

### 10.0 Conclusion

From the foregoing, it will be appreciated that specific embodiments of the disclosed technology have been described herein for purposes of illustration, but that various modifications may be made without deviating from the technology. For example, therapy signals described herein can be delivered at combinations of parameter values within the foregoing ranges at values that are not expressly disclosed herein. Certain aspects of the technology described in the context of particular embodiments may be combined or eliminated in other embodiments. For example, the therapy signal can be monophasic with a passive charge elimination phase. In some embodiments, the foregoing techniques can be used to address patient deficits than pain. Further, while advantages associated with certain embodiments of the disclosed technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

The use of “and/or,” as in “A and/or B” refers to A alone, B alone, and both A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Where a range of values is provided, it is understood that each intervening value, to the tenth of the unit of the lower limit unless the context clearly dictates otherwise, to between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Each of the patents and patent application publications referenced herein are incorporated by reference in their entireties and for all purposes. However, to the extent any patent or patent application publication conflicts with the present disclosure, the present disclosure controls.

What is claimed is:

1. A method for lowering blood glucose levels in a patient using spinal column stimulation, the method comprising:
programming a signal generator to:
generate an electrical signal having an amplitude at or above a motor threshold of the patient, and
deliver the electrical signal to a spinal column of the patient via an implanted signal delivery device positioned proximate a target neural population associated with the patient's abdomen and/or lower extremities,
wherein the electrical signal induces motor contractions in the patient's abdomen and/or lower extremities to reduce a blood glucose level in the patient.

2. The method of claim 1, wherein the electrical signal has a frequency within a frequency range of from about 0.1 Hz to about 2 Hz.

3. The method of claim 1, wherein the electrical signal has a frequency range of from about 500 Hz to about 10 kHz.

4. The method of claim 1, wherein the electrical signal has a frequency range of from about 10 Hz to about 50 Hz.

5. The method of claim 1, wherein the electrical signal is delivered in periods of from about 1 second to about 10 seconds followed by a quiescent period of from about 1 second to about 10 seconds.

6. The method of claim 1, wherein an amplitude of the electrical signal is ramped from below the perception threshold to between about 100-150% of the motor threshold of the patient.

7. The method of claim 6, wherein the electrical signal is ramped from about 90% of the perception threshold to about 150% of the motor threshold of the patient.

8. The method of claim 1, wherein programming the signal generator to generate and deliver the electrical signal includes programming the signal generator to generate and deliver the electrical signal during discrete stimulation sessions.

9. The method of claim 8, wherein the discrete stimulation sessions include two or more stimulation sessions per day.

10. The method of claim 8, wherein each stimulation session has a duration of between about 15 minutes and about 45 minutes.

11. The method of claim 1, wherein programming the signal generator to generate and deliver the electrical signal includes programming the signal generator to:
receive an indication of an activity level of a patient;
in response to the activity level of the patient being below a predetermined threshold, generate and deliver the electrical signal, and
in response to the activity level of the patient exceeding the predetermined threshold, ceasing generating and delivering the electrical signal.

12. The method of claim 1, wherein programming the signal generator is performed in response to the patient having an elevated blood glucose level.

13. The method of claim 1, wherein the electrical signal reduces the blood glucose level in the patient in an insulin-independent manner.

14. A method of decreasing blood glucose levels in a patient, the method comprising:
delivering electrical stimulation to a target neural population within a spinal column of the patient, wherein the target neural population is associated with motor control of a lower extremity or an abdomen of the patient, and wherein the electrical stimulation has an amplitude at or above a motor threshold,
wherein the electrical stimulation induces motor contractions in the lower extremity and/or abdomen of the patient to uptake glucose from the bloodstream of the patient.

15. The method of claim 14, further comprising:
measuring a blood glucose level of the patient;
comparing the measured blood glucose level of the patient to a baseline glucose level of the patient; and
adjusting one or more signal parameters of the electrical stimulation based on the comparison.

16. The method of claim 14, further comprising:
monitoring an activity level of the patient;
in response to the activity level of the patient exceeding a predetermined threshold indicating the patient is in an active state, ceasing deliver of the electrical stimulation; and
in response to the activity level returning to below the predetermined threshold, resuming deliver of the electrical stimulation.

17. The method of claim 14, further comprising:
determining the motor threshold of the patient;
delivering the electrical stimulation at an amplitude between about 100% and 150% of the motor threshold; and
adjusting the amplitude based on at least in part on the motor contractions.

18. The method of any of claim 16, wherein the motor contractions are detected using electromyography (EMG) sensors placed on the lower extremity or the abdomen of the patient.

19. The method of 14, wherein the electrical stimulation is delivered through electrodes implanted in an epidural space of the spinal column of the patient.
